(19) United States
(12) Patent Application Publication
(10) Pub. No.: US 2008/0226714 A1
(43) Pub. Date: Sep. 18, 2008

(54) SUSTAINED-RELEASE TABLET FORMULATIONS OF PIPERAZINE-PIPERIDINE ANTAGONISTS AND AGONISTS OF THE 5-HT1A RECEPTOR HAVING ENHANCED INTESTINAL DISSOLUTION

(75) Inventors: Hong Wen, Westfield, NJ (US); Chimanlall Goolcharran, Hopewell Junction, NY (US); Krishnendu Ghosh, Sparkill, NY (US); Arwinder Nagi, Thiells, NY (US)

Correspondence Address: WilmerHale/Wyeth, 60 STATE STREET, BOSTON, MA, 02109, US

(73) Assignee: WYETH, Madison, NJ (US)

(21) Appl. No.: 12/032,003

(22) Filed: Feb. 15, 2008

Related U.S. Application Data
(60) Provisional application No. 60/901,912, filed on Feb. 16, 2007.

(51) Int. Cl. A61K 31/496; A61K 9/22

(52) U.S. Cl. 424/468; 514/253.06

(57) ABSTRACT

The present invention relates to sustained-release tablet formulations of piperazine-piperidine compounds, which can be useful in treating central nervous system disorders; to processes for their preparation; and to methods of using them.

### CROSS REFERENCE TO RELATED APPLICATIONS

This application claims the benefit of priority under 35 U.S.C. §119(e) to U.S. Patent Application Ser. No. 60/901,912 filed on Feb. 16, 2007.

### FIELD OF THE INVENTION

The present invention relates to sustained-release tablet formulations of piperazine-piperidine compounds, which can be useful in treating central nervous system disorders; to processes for their preparation; and to methods of using them.

### BACKGROUND OF THE INVENTION

Certain N-aryl-piperazine derivatives possess pharmaceutical activity. In particular, certain N-aryl piperazine derivatives act on the central nervous system (CNS) by binding to 5-HT receptors. In pharmacological testing, it has been shown that the certain N-aryl-piperazine derivatives bind to receptors of the 5-HT_{1A} type. Many of the N-aryl piperazine derivatives exhibit activity as 5-HT_{1A} antagonists. See, for example, W. C. Childers, et al., J. Med. Chem., 48: 3467-3470 (2005), U.S. Pat. Nos. 6,465,482, 6,127,357, 6,469,007, and 6,586,436, and PCT Publication No. WO 97/03982.

Pharmaceutical compounds that interact with the 5-HT_{1A} receptor are useful to treat a wide variety of central nervous system disorders, such as cognition disorders, anxiety disorders, and depression. Certain piperazine-piperidine compounds have shown utility as 5-HT_{1A} receptor antagonists, agonists and partial antagonist/agonists. See, for example, U.S. Patent Publication No. 2007/0027160, filed Jun. 9, 2006, entitled “piperazine-Piperidine Antagonists And Agonists Of The 5-HT_{1A} Receptor”. For example, some diquinoline-substituted piperazine-piperidine compounds, such as 5-fluoro-8-{4-[4-(6-methoxyquinolin-8-yl)piperazin-1-yl]piperidin-1-yl}quinoline, have been demonstrated to be antagonists of the 5-HT_{1A} receptor. During development of 5-fluoro-8-{4-[4-(6-methoxyquinolin-8-yl)piperazin-1-yl]piperidin-1-yl}quinoline, it was discovered that the compound has a pH-dependent solubility. Over the pH range of about 2.4 to about 8.9, the solubility ranges from about 2.2 mg/mL to about 36 ng/mL, respectively. The compound (free base) has very low water solubility (approximately 0.04 μg/mL) and is nearly insoluble around the neutral pH of the lower gastrointestinal tract (less than about 1 μg/mL at pH levels greater than about 6). Although the trisuccinate salt of 5-fluoro-8-{4-[4-(6-methoxyquinolin-8-yl)piperazin-1-yl]piperidin-1-yl}quinoline was found to have higher aqueous solubility than the free base at approximately 1 mg/mL, it still has a pH-dependent solubility with the solubility being at the sub-microgram per milliliter level at pH greater than about six. Permeability characteristics of the molecule in animal studies indicated that this compound has high permeability. Therefore, the rate of systemic input of 5-fluoro-8-{4-[4-(6-methoxyquinolin-8-yl)piperazin-1-yl]piperidin-1-yl}quinoline will be dependent on the dissolution or solubilization of the compound. In addition, in preclinical studies 5-fluoro-8-{4-[4-(6-methoxyquinolin-8-yl)piperazin-1-yl]piperidin-1-yl}quinoline has demonstrated a short half-life in Sprague-Dawley rats (t_{1/2} of about one hour at 0.3 mg/kg IV bolus). A controlled-release formulation to reduce the dose frequency of 5-fluoro-8-{4-[4-(6-methoxyquinolin-8-yl)piperazin-1-yl]piperidin-1-yl}quinoline would be beneficial for improving compliance and convenience. However, this type of formulation presents the challenge of maintaining the dissolution of the compound in the lower gastrointestinal tract where solubility is very low.

### SUMMARY OF THE INVENTION

In one aspect, the present invention provides a sustained-release tablet formulation for oral administration comprising:

(a) a compound of formula (I), or a pharmaceutically acceptable salt thereof,

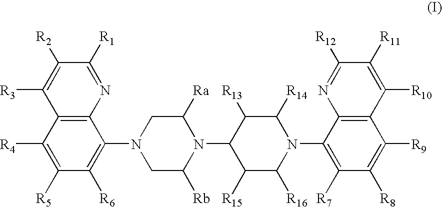

wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, SO₂R₂₅, SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is H, linear or branched (C₁-C₆)-alkyl, (C₁-C₆)-haloalkyl, (C₂-C₆)-alkenyl, or (C₂-C₆)-alkynyl;

(b) at least one organic acid;

(c) at least one release controller;

(d) at least one filler; and

(e) at least one lubricant.

In certain embodiments, in the compound of formula (I), R₅ is —H, (C₁-C₆)-alkyl, —OR₂₅, halogen, or —CF₃; and R₉ is —H, (C₁-C₆)-alkyl, —OR₂₅, halogen, —CF₃, —NO₂, or —CN. In certain other embodiments, the compound of formula (I) is selected from the group consisting of:
6-methoxy-8-[4-(1-quinolin-8-yl-piperidin-4-yl)-piperazin-1-yl]-quinoline;
6-fluoro-8-[4-(1-quinolin-8-yl-piperidin-4-yl)-piperazin-1-yl]-quinoline;
5-fluoro-8-[4-(1-quinolin-8-yl-piperidin-4-yl)-piperazin-1-yl]-quinoline;
7-fluoro-8-(4-(4-(6-methoxyquinolin-8-yl)piperazin-1-yl)piperidin-1-yl)quinoline;
6-fluoro-8-{4-[1-(8-fluoroquinolin-7-yl)piperidin-4-yl]piperazin-1-yl}quinoline;
3-trifluoromethyl-8-(4-(4-(6-methoxyquinolin-8-yl)piperazin-1-yl)piperidin-1-yl)quinoline;
6-methoxy-8-(4-(1-(quinolin-8-ylmethyl)piperidin-4-yl)piperazin-1-yl)quinoline;
5-fluoro-4-methoxy-8-(4-(4-(6-methoxyquinolin-8-yl)piperazin-1-yl)piperidin-1-yl)-2-(trifluoromethyl)quinoline;
5-fluoro-8-(4-(4-(6-methoxyquinolin-8-yl)piperazin-1-yl)piperidin-1-yl)quinoline;
5-fluoro-8-(4-(4-(6-methoxyquinolin-8-yl)piperazin-1-yl)piperidin-1-yl)quinoline trisuccinate;
8-[4-(1-quinolin-8-yl-piperidin-4-yl)-piperazin-1-yl]-quinoline;
6-chloro-8-[4-(4-(6-chloro)-quinolin-8-yl-piperidin-1-yl)-piperazin-1-yl]-quinoline;
6-fluoro-8-[4-(4-(6-chloro)-quinolin-8-yl-piperidin-1-yl)-piperazin-1-yl]-quinoline;
5-chloro-8-[4-(1-quinolin-8-yl-piperidin-4-yl)-piperazin-1-yl]-quinoline;
2-methyl-8-[4-(1-quinolin-8-yl-piperidin-4-yl)-piperazin-1-yl]-quinoline;
6-chloro-8-[4-(1-quinolin-8-yl-piperidin-4-yl)-piperazin-1-yl]-quinoline;
8-[4-(1-quinolin-8-yl-piperidin-4-yl)-piperazin-1-yl]-5-trifluoromethyl-quinoline;
5-methoxy-8-[4-(1-quinolin-8-yl-piperidin-4-yl)-piperazin-1-yl]-quinoline;
5-fluoro-8-[4-(4-quinolin-8-yl-piperazin-1-yl)-piperidin-1-yl]-quinoline;
6-methoxy-8-[4-(2-methylquinolin-8-yl-piperidin-4-yl)-piperazin-1-yl]-quinoline;
6-fluoro-8-(4-(1-(2-methylquinolin-8-yl)piperidin-4-yl)piperazin-1-yl)quinoline;
6-methoxy-8-[4-(3-methylquinolin-8-yl-piperidin-4-yl)-piperazin-1-yl]-quinoline;
6-methoxy-8-(4-(1-(4-methylquinolin-8-yl)piperidin-4-yl)piperazin-1-yl)quinoline;
6-methoxy-8-(4-(1-(2,4-dimethylquinolin-8-yl)piperidin-4-yl)piperazin-1-yl)quinoline;
6-methoxy-8-(4-(1-(2,4-dimethyl-5-fluoroquinolin-8-yl)piperidin-4-yl)piperazin-1-yl)quinoline;
6-methoxy-8-(4-(1-(2-(trifluoromethyl)quinolin-8-yl)piperidin-4-yl)piperazin-1-yl)quinoline;
6-fluoro-8-(4-(1-(5-fluoroquinolin-8-yl)piperidin-4-yl)piperazin-1-yl)quinoline;
6-methoxy-8-(4-(1-(6-bromoquinolin-8-yl)piperidin-4-yl)piperazin-1-yl)quinoline;
6-methoxy-8-(4-(1-(6-fluoroquinolin-8-yl)piperidin-4-yl)piperazin-1-yl)quinoline;
6-fluoro-8-(4-(1-(7-fluoroquinolin-8-yl)piperidin-4-yl)piperazin-1-yl)quinoline;
6-methoxy-8-{4-[1-(8-fluoroquinolin-7-yl)piperidin-4-yl]piperazin-1-yl}quinoline;
6-methoxy-8-{4-[1-(2-trifluoromethyl-4-methoxyquinolin-7-yl)piperidin-4-yl]piperazin-1-yl}quinoline;
6-methoxy-8-(4-(1-(2-trifluoromethyl-4-methoxyquinolin-8-yl)piperidin-4-yl)piperazin-1-yl)quinoline;
5-fluoro-8-(4-(4-(6-methoxyquinolin-8-yl)piperazin-1-yl)piperidin-1-yl)-2-trifluoromethylquinoline;
5-fluoro-8-(4-(4-(6-methoxyquinolin-8-yl)piperazin-1-yl)piperidin-1-yl)-3-trifluoromethylquinoline;
5-fluoro-8-(4-(4-(6-methoxyquinolin-8-yl)piperazin-1-yl)piperidin-1-yl)-4-trifluoromethylquinoline;
2,5-difluoro-8-(4-(4-(6-methoxyquinolin-8-yl)piperazin-1-yl)piperidin-1-yl)quinoline;
3,5-difluoro-8-(4-(4-(6-methoxyquinolin-8-yl)piperazin-1-yl)piperidin-1-yl)quinoline; and
4,5-difluoro-8-(4-(4-(6-methoxyquinolin-8-yl)piperazin-1-yl)piperidin-1-yl)quinoline.

In yet another embodiment, the compound of formula (I) is 5-fluoro-8-{4-[4-(6-methoxyquinolin-8-yl)piperazin-1-yl]piperidin-1-yl}quinoline trisuccinate.

In another aspect, the present invention provides a sustained-release tablet formulation for oral administration comprising:

(a) a compound of formula (I), or a pharmaceutically acceptable salt thereof, as described herein;

(b) at least one organic acid;

(c) at least one release controller;

(d) at least one filler; and

(e) at least one lubricant,

Characterized in that the formulation releases at least about 25% by weight of the compound of formula (I) or a pharmaceutically acceptable salt thereof, after 8 hours in a dissolution test apparatus having a rotation speed of 75 rpm, and a single stage dissolution medium containing pH 4.5 buffer at 37° C. In one or more embodiments, the test apparatus is Apparatus 2 described in the United States Pharmacopoeia (USP29-NF24, page 2673).

In a further aspect, the present invention provides a sustained-release tablet formulation for oral administration comprising:

(a) a compound of formula (I), or a pharmaceutically acceptable salt thereof, as described herein;

(b) at least one organic acid;

(c) at least one release controller;

(d) at least one filler; and

(e) at least one lubricant,

characterized in that the formulation releases at least 45% by weight of the compound of formula (I) or a pharmaceutically acceptable salt thereof, after 8 hours in a dissolution test apparatus having a rotation speed of 75 rpm, and a single stage dissolution medium containing pH 4.5 buffer at 37° C. In one or more embodiments, the dissolution test apparatus is Apparatus 2 described in the United States Pharmacopoeia (USP29-NF24, page 2673).

In certain embodiments, the formulation releases at least 15% by weight of the compound of formula (I) or a pharmaceutically acceptable salt thereof after 2 hours under the one stage dissolution condition as described above. In certain other embodiments, the formulation releases less than 40% by weight, or less than 20% by weight of the compound of formula (I) or a pharmaceutically acceptable salt thereof after 2 hours under the one stage dissolution condition.

In certain embodiments, the formulation releases less than 60% by weight, or less than 40% by weight of the compound of formula (I) or a pharmaceutically acceptable salt thereof after 4 hours under the one stage dissolution condition. In some cases, less than 70% by weight, or less than 50% by weight of the compound of formula (I) or a pharmaceutically acceptable salt thereof, is released after 6 hours under the one stage dissolution condition as described above.

In yet another aspect, the present invention provides a sustained-release tablet formulation for oral administration comprising:

(a) a compound of formula (I), or a pharmaceutically acceptable salt thereof, as described herein;

(b) at least one organic acid;

(c) at least one release controller;

(d) at least one filler; and

(e) at least one lubricant,

characterized in that the formulation releases at least 60% by weight of the compound of formula (I) or a pharmaceutically acceptable salt thereof, after 8 hours in a dissolution test apparatus having a rotation speed of 75 rpm, and a two-stage dissolution medium containing (i) pH 1 buffer at 37° C. for two hours, and followed by (ii) pH 6.5 buffer with 1% SLS for additional 6 hours at 37° C. In one or more embodiments, the dissolution test apparatus is Apparatus 2 described in the United States Pharmacopoeia (USP29-NF24, page 2673).

In certain embodiments, the formulation releases at least 35% by weight of the compound of formula (I) or a pharmaceutically acceptable salt thereof after 2 hours under the two-stage dissolution condition as described above. In certain other embodiments, the formulation releases less than 55% by weight, or less than 40% by weight of the compound of formula (I) or a pharmaceutically acceptable salt thereof after 2 hours under the two-stage dissolution condition.

In certain embodiments, the formulation releases less than 70% by weight, or less than 50% by weight of the compound of formula (I) or a pharmaceutically acceptable salt thereof after 4 hours under the two-stage dissolution condition. In some cases, less than 80% by weight, or less than 60% by weight of the compound of formula (I) or a pharmaceutically acceptable salt thereof, is released after 6 hours under the two-stage dissolution condition as described above.

In certain embodiments, the organic acid is citric acid, ascorbic acid, aspartic acid, glutamic acid, tartaric acid, succinic acid, malic acid, erythorbic acid, propionic acid, lactic acid, oleic acid, fumaric acid, benzoic acid, or alginic acid.

In certain other embodiments, the release controller is hydroxypropyl methylcellulose (HPMC), hydroxypropyl cellulose, hydroxyethyl cellulose, or hydroxypropyl methylcellulose phthalate. In some cases, the release controller is one or more HPMC selected from Methocel® K4M Premium CR, Methocel® K100M Premium CR, and Methocel® K100LV Premium CR.

In certain embodiments, the filler is microcrystalline cellulose, silicified microcrystalline cellulose, lactose, calcium carbonate, calcium sulfate, calcium phosphate, sodium chloride, maltodextrin, dextrose, fructose, maltose, mannitol, starch, sucrose, or kaolin.

In certain other embodiments, the lubricant is magnesium stearate, calcium stearate, stearic acid, talc, hydrogenated vegetable oils, polyethylene glycol, or colloidal silicon dioxide.

In certain embodiments, the sustained-release tablet formulation contains from about 0.1 mg to about 100 mg of the compound of formula (I), or a pharmaceutically acceptable salt thereof. In some cases, the formulation contains from about 0.5 mg to about 25 mg of the compound of formula (I), or a pharmaceutically acceptable salt thereof. In certain other embodiments, the compound of formula (I), or a pharmaceutically acceptable salt thereof, makes up about 0.02% to about 20% by weight of the formulation, expressed in the form of its free base.

In certain embodiments, the organic acid makes up about 2% to about 20% by weight of the formulation, for example, about 5% to about 15% by weight of the formulation. In certain other embodiments, the release controller makes up about 10% to about 60% by weight of the formulation, for example, about 30% to about 50% by weight of the formulation.

In certain embodiments, the filler makes up about 25% to about 65% by weight of the formulation. In certain other embodiments, the lubricant makes up about 0.1% to about 5% by weight of the formulation.

In one aspect, the present invention provides a method for treating a 5-HT_{1A}-related disorder to a patient in need thereof, the method comprising administering to the patient a therapeutically effective amount of a sustained-release tablet formulation as described hereinabove. In certain embodiments, the 5-HT_{1A}-related disorder is a cognition-related disorder or an anxiety-related disorder. In certain other embodiments, the cognition-related disorder is dementia, Parkinson's disease, Huntington's disease, Alzheimer's disease, cognitive deficits associated with Alzheimer's disease, mild cognitive impairment, or schizophrenia. In certain embodiments, the anxiety-related disorder is attention deficit disorder, obsessive compulsive disorder, substance addiction, withdrawal from substance addiction, premenstrual dysphoric disorder, social anxiety disorder, anorexia nervosa, or bulimia nervosa.

In another aspect, the present invention provides a process for the preparation of a sustained-release tablet formulation as described hereinabove, which process comprises mixing: a compound of formula (I) or a pharmaceutically acceptably salt thereof, at least one organic acid; at least one release controller; at least one filler; at least one lubricant; followed by pressing into tablet. In certain embodiments, the process may further comprise film coating of the tablet.

In one or more embodiments, the invention provides the use of a formulation as described herein in the manufacture of a medicament for treating a 5HT_{1A}-related disorder.

### BRIEF DESCRIPTION OF THE DRAWINGS

FIG. 1 is a flow chart showing a process for preparing sustained-release tablet formulations of the present invention.

### DETAILED DESCRIPTION

The term “(C₁-C₆)-alkyl” as used herein refers to a linear or branched, saturated hydrocarbon having from 1 to 6 carbon atoms. Representative (C₁-C₆)-alkyl groups include, but are not limited to, methyl, ethyl, propyl, isopropyl, butyl, sec-butyl, tert-butyl, pentyl, isopentyl, neopentyl, hexyl, isohexyl, and neohexyl. In one embodiment, the (C₁-C₆)-alkyl group is optionally substituted with one or more of the following groups: halogen, N₃, —NO₂, —CN, —OR′, —SR′, —SO₂R′, SO₂N(R′)₂, N(R′)₂, COR′, CO₂R′, NR′CO₂R′, NR′COR′, —NR′CONR′, or —CON(R′)₂, wherein each R′ is independently hydrogen or unsubstituted (C₁-C₆)-alkyl.

The term “(C₂-C₆)-alkenyl” as used herein refers to a linear or branched hydrocarbon having from 2 to 6 carbon atoms and having at least one carbon-carbon double bond. In one embodiment, the (C₂-C₆)-alkenyl has one or two double bonds. The (C₂-C₆)-alkenyl moiety may exist in the E or Z conformation and the compounds of the present invention include both conformations. In one embodiment, the (C₂-C₆)-alkenyl group is optionally substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

The term “(C₂-C₆)-alkynyl” as used herein refers to a linear or branched hydrocarbon having from 2 to 6 carbon atoms and having at least one carbon-carbon triple bond. In one embodiment, the (C₂-C₆)-alkenyl group is optionally substituted with one or more of the following groups: halogen, —N₃, —NO₂, —CN, —OR′, —SR′, —SO₂R′, —SO₂N(R′)₂, —N(R′)₂, —COR′, —CO₂R′, —NR′CO₂R′, —NR′COR′, —NR′CONR′, or —CON(R′)₂, wherein each R′ is independently hydrogen or unsubstituted (C₁-C₆)-alkyl.

The term “(C₁-C₆)-haloalkyl” refers to a C₁-C₆ alkyl group, as defined above, wherein one or more of the C₁-C₆ alkyl group's hydrogen atoms has been replaced with —F, —Cl, —Br or −I. Representative examples of an alkylhalo group include, but are not limited to, —CH₂F, —CCl₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(CI)CH₂CH₃, —CH(F)CH₂CH₃, —C(CH₃)₂(CH₂Cl), —CH₂CH₂CH₂CH₂CH₂CH₂Br, and —CH₂CH₂CH₂CH₂CH₂CH₂I.

The term “administer”, “administering”, or “administration”, as used herein refers to either directly administering a compound or pharmaceutically acceptable salt of the compound or a formulation to an animal, or administering a prodrug derivative or analog of the compound or pharmaceutically acceptable salt of the compound or formulation to the animal, which can form an equivalent amount of active compound within the animal's body.

The term “animal” as used herein includes, without limitation, a human, mouse, rat, guinea pig, dog, cat, horse, cow, pig, monkey, chimpanzee, baboon, or rhesus. In one embodiment, the animal is a mammal. In another embodiment, the animal is a human.

The term “aryl” as used herein refers to an aromatic species containing 1 to 3 aromatic rings, either fused or linked. In one embodiment, the aromatic species contains 1 to 3 aromatic rings; in another embodiment, the aromatic species contains 1 to 2 aromatic rings; in a further embodiment, the aromatic ring contains 1 aromatic ring. In one embodiment, the aryl group is optionally substituted with one or more of the following groups: —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “conditions effective to” as used herein refers to synthetic reaction conditions that will be apparent to those skilled in the art of synthetic organic chemistry.

The term “cyclic group” as used herein includes a cycloalkyl group and a heterocyclic group. Any suitable ring position of the cyclic group may be covalently linked to the defined chemical structure. In one embodiment, the cyclic group is optionally substituted with one or more of the following groups: —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “cycloalkyl group” as used herein refers to a three- to seven-membered saturated or partially unsaturated carbon ring. In one embodiment, the cycloalkyl group refers to a three to seven-membered ring; in another embodiment, the cycloalkyl group refers to a three to six-membered ring; in a further embodiment, the cycloalkyl group refers to a three to five-membered ring; in yet another embodiment, the cycloalkyl group refers to a three to four-membered ring; and in one embodiment the cycloalkyl group refers to a three-membered ring. Any suitable ring position of the cycloalkyl group may be covalently linked to the defined chemical structure. Exemplary cycloalkyl groups include cyclopropyl, cyclobutyl, cyclopentyl, cyclohexyl, and cycloheptyl. In one embodiment, the cycloalkyl group is optionally substituted with one or more of the following groups: —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “effective amount” as used herein refers to an amount of a compound or pharmaceutically acceptable salt of a compound that, when administered to an animal, is effective to prevent, to at least partially ameliorate, or to cure, a condition from which the animal suffers or is suspected to suffer.

The term “halogen” as used herein refers to fluorine, chlorine, bromine, and iodine.

The term “heterocyclic group” as used herein refers to a three- to seven-membered saturated, partially saturated, or unsaturated cycloalkyl group in which one to four of the ring carbon atoms have been independently replaced with a N, O, or S atom. Any suitable ring position of the heterocyclic group may be covalently linked to the defined chemical structure.

In one embodiment, the heterocyclic group refers to a three to seven-membered ring; in another embodiment, the heterocyclic group refers to a three to six-membered ring; in a further embodiment, the heterocyclic group refers to a three to five-membered ring; in yet another embodiment, the heterocyclic group refers to a three to four-membered ring; and in one embodiment the heterocyclic group refers to a three-membered ring. Exemplary heterocyclic groups include, but are not limited to, azepanyl, azetidinyl, aziridinyl, furanyl, furazanyl, homopiperazinyl, imidazolidinyl, imidazolinyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazolyl, pyridoimidazolyl, pyridothiazolyl, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, and triazolyl. In one embodiment, the heterocyclic group is optionally substituted with one or more of the following groups: —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “isolated and purified” as used herein refers to separate from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt of the compound by weight of the isolate.

The term “pharmaceutically acceptable salt”, as used herein, refers to salts derived from organic and inorganic acids of a compound of the present invention. Exemplary salts include, but are not limited to sulfate, citrate, acetate, oxalate, chloride, hydrochloride, bromide, hydrobromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, succinate, fumarate, maleate, malonate, mandelate, malate, phthalate, and pamoate. The term “pharmaceutically acceptable salt” as used herein also refers to a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. The term “pharmaceutically acceptable salt” also includes hydrates of a compound of the present invention.

The term “phenyl” as used herein refers to a substituted or unsubstituted phenyl group. In one embodiment, the phenyl group is optionally substituted with one or more of the following groups: —V-halogen, —V—N₃, —V—NO₂, —V—CN, —V—OR′, —V—SR′, —V—SO₂R′, —V—SO₂N(R′)₂, —V—N(R′)₂, —V—COR′, —V—CO₂R′, —V—NR′CO₂R′, —V—NR′COR′, —V—NR′CONR′, or —V—CON(R′)₂, wherein each R′ is independently hydrogen or unsubstituted (C₁-C₆)-alkyl; and wherein each V is independently a bond or (C₁-C₆)-alkyl.

The term “substantially free of its corresponding opposite enantiomer” as used herein means that the compound contains no more than about 10% by weight of its corresponding opposite enantiomer. In other embodiments, the compound that is substantially free of its corresponding opposite enantiomer contains no more than about 5%, no more than about 1%, no more than about 0.5%, or no more than about 0.1% by weight of its corresponding opposite enantiomer. An enantiomer that is substantially free of its corresponding opposite enantiomer includes a compound that has been isolated and purified or has been prepared substantially free of its corresponding opposite enantiomer.

The term “5-HT_{1A}-related disorder” as used herein refers to a condition that is mediated through the 5-HT_{1A} receptor. In some embodiments, a 5-HT_{1A}-related disorder is a condition for which it would be beneficial to prevent activation of the 5-HT_{1A} receptor. In other embodiments, a 5-HT_{1A}-related disorder is a condition for which it would be beneficial to activate the 5-HT_{1A} receptor. In one embodiment, a 5-HT_{1A}-related disorder affects the central nervous system (i.e., a CNS-related disorder). Exemplary 5-HT_{1A}-related disorders include, without limitation, depression, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder, pediatric depression, child abuse induced depression and postpartum depression; bipolar disorders or manic depression, for example, bipolar I disorder, bipolar II disorder and cyclothymic disorder; conduct disorder; disruptive behavior disorder; disorders of attention and learning such as attention deficit hyperactivity disorder (ADHD) and dyslexia; behavioral disturbances associated with mental retardation, autistic disorder, pervasive development disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia, substance-induced psychotic disorder, shared psychotic disorder, and psychotic disorder due to a general medical condition; delirium, dementia, and amnestic and other cognitive or neurodegenerative disorders, such as Parkinson's disease (PD), Huntington's disease (HD), Alzheimer's disease, senile dementia, dementia of the Alzheimer's type, mild cognitive impairment (MCI), memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; cognitive deficits associated with neurological conditions including, for example, Parkinson's disease (PD), Huntington's disease (HD), Alzheimer's disease; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependencies on, or addictions to, alcohol, heroin, cocaine, benzodiazepines, nicotine, or phenobarbitol); behavioral addictions such as an addiction to gambling; and ocular disorders such as glaucoma and ischemic retinopathy; sexual dysfunction associated with drug treatment (e.g., sexual dysfunction associated with SSRI's).

One nonlimiting example of a 5-HT_{1A}-related disorder is a cognition-related disorder (e.g., cognitive dysfunction). Exemplary cognition-related disorders include, without limitation, mild cognitive impairment (MCI), dementia, delirium, amnestic disorder, Alzheimer's disease, Parkinson's disease, Huntington's disease, memory disorders including memory deficits associated with depression, senile dementia, dementia of Alzheimer's disease, cognitive deficits or cognitive dysfunction associated with neurological conditions including, for example, Parkinson's disease (PD), Huntington's disease (HD), Alzheimer's disease, depression and schizophrenia (and other psychotic disorders such as paranoia and mano-depressive illness); cognitive dysfunction in schizophrenia, disorders of attention and learning such as attention deficit disorders (e.g., attention deficit hyperactivity disorder (ADHD)) and dyslexia, cognitive dysfunction associated with developmental disorders such as Down's syndrome and Fragile X syndrome, loss of executive function, loss of learned information, vascular dementia, schizophrenia, cognitive decline, neurodegenerative disorder, and other dementias, for example, due to HIV disease, head trauma, Parkinson's disease, Huntington's disease, Pick's disease, Creutzfeldt-Jakob disease, or due to multiple etiologies. Cognition-related disorders also include, without limitation, cognitive dysfunction associated with MCI and dementias such as Lewy Body, vascular, and post stroke dementias. Cognitive dysfunction associated with surgical procedures, traumatic brain injury or stroke may also be treated in accordance with the present invention.

Another nonlimiting example of a 5-HT_{1A}-related disorder is an anxiety-related disorder. Exemplary anxiety-related disorders include, without limitation, generalized anxiety disorder, attention deficit disorder, attention deficit hyperactivity disorder, obsessive compulsive disorder, substance addiction, withdrawal from drug, alcohol or nicotine addiction, panic disorder, panic attacks, post-traumatic stress disorder, premenstrual dysphoric disorder, social anxiety disorder, eating disorders such as anorexia nervosa and bulimia nervosa, vasomotor flushing, and phobias, including social phobia, agoraphobia, and specific phobias. Substance addition includes, without limitation, drug, alcohol or nicotine addiction.

“Cₘₐₓ,”. “Tₘₐₓ,” and “AUC” values reported herein, unless stated as being “mean” values, refer to the values observed in an individual patient. Moreover, Cₘₐₓ, Tₘₐₓ, and AUC values, unless otherwise stated, may be values observed at steady state when dosing at regular time intervals (e.g., every 12 hours) for multiple days (e.g., multiple dose administration) or values for a single dose administration.

Various embodiments of the invention are described herein. It will be readily understood by a person of skill in the art that the embodiments may be combined.

The sustained-release tablet formulations described herein include:

(a) a compound of formula (I), or a pharmaceutically acceptable salt thereof

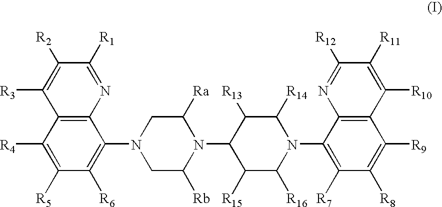

wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;

Rₐ and R_{b} are each independently —H or —CH₃; and

R₂₅ is —H, linear or branched (C₁-C₆)-alkyl, (C₁-C₆)-haloalkyl, (C₂-C₆)-alkenyl, or (C₂-C₆)-alkynyl;

(b) at least one organic acid;

(c) at least one release controller;

(d) at least one filler; and

(e) at least one lubricant.

In one embodiment, R₁ is —H, (C₁-C₆)-alkyl, —OR₂₅, halogen, or —CF₃. In another embodiment, R₁ is —H, (C₁-C₆)-alkyl, —OR₂₅, halogen, or —CF₃ and one of R₁₃, R₁₄, R₁₅, and

R₁₆ is —H, (C₁-C₆)-alkyl, —OR₂₅, or halogen. In a further embodiment, R₁ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₇, R₈, R₉, R₁₀, R₁₁, and R₁₂ are each hydrogen. In yet another embodiment, R₁ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₂, R₃, R₄, R₅, R₆, R₇, R₈, R₉, R₁₀, R₁₁, and R₁₂ are each hydrogen. In one embodiment, R₁ is —H, (C₁-C₆)-alkyl, —OR₂₅, halogen or —CF₃ and R₂, R₃, R₄, R₅, R₆, R₇, R₈, R₉, R₁₀, R₁₁, R₁₂, R₁₃, R₁₄, R₁₅, and R₁₆ are each hydrogen.

In one embodiment, R₄ is —H, (C₁-C₆)-alkyl, —OR₂₅, halogen, or —CF₃. In another embodiment, R₄ is —H, (C₁-C₆)-alkyl, —OR₂₅, halogen, or —CF₃ and one of R₁₃, R₁₄, R₁₅, and R₁₆ is —H, (C₁-C₆)-alkyl, —OR₂₅, or halogen. In a further embodiment, R₄ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₇, R₈, R₉, R₁₀, R₁₁, and R₁₂ are each hydrogen. In yet another embodiment, R₄ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₁, R₂, R₃, R₅, R₆, R₇, R₈, R₉, R₁₀, R₁₁, and R₁₂ are each hydrogen. In one embodiment, R₄ is —H, (C₁-C₆)-alkyl, —OR₂₅, halogen or —CF₃ and R₁, R₂, R₃, R₅, R₆, R₇, R₈, R₉, R₁₀, R₁₁, R₁₂, R₁₃, R₁₄, R₁₅, and R₁₆ are each hydrogen.

In one embodiment, R₅ is —H, (C₁-C₆)-alkyl, —OR₂₅, halogen, or —CF₃. In another embodiment, R₅ is —H, (C₁-C₆)-alkyl, —OR₂₅, halogen, or —CF₃ and one of R₁₃, R₁₄, R₁₅, and R₁₆ is —H, (C₁-C₆)-alkyl, —OR₂₅, or halogen. In a further embodiment, R₅ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and R₇, R₈, R₉, R₁₀, R₁₁, and R₁₂ are each hydrogen. In yet another embodiment, R₅ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen; and R₁, R₂, R₃, R₄, R₆, R₇, R₈, R₉, R₁₀, R₁₁, and R₁₂ are each hydrogen. In one embodiment, R₅ is —H, (C₁-C₆)-alkyl, —OR₂₅, halogen or —CF₃ and R₁, R₂, R₃, R₄, R₆, R₇, R₈, R₉, R₁₀, R₁₁, R₁₂, R₁₃, R₁₄, R₁₅, and R₁₆ are each hydrogen. In a further embodiment, one of R₁₃, R₁₄, R₁₅, and R₁₆ is —H, (C₁-C₆)-alkyl, halogen, —CF₃, or —OR₂₅; R₅ is —H, (C₁-C₆)-alkyl, —OR₂₅, halogen, or —CF₃; and the remaining Rₐ, R_{b}, and R₁₋₁₆ are each hydrogen.

In one embodiment, R₈ is —H, (C₁-C₆)-alkyl, —OR₂₅, halogen, —CF₃, —NO₂ or —CN. In another embodiment, R₈ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; Rₐ and R_{b} are each independently —H or —CH₃; and the remaining Rₐ, R_{b}, and R₁₋₇ and R₉₋₁₆ are each hydrogen. In a further embodiment, R₈ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and the remaining Rₐ, R_{b}, and R₁₋₇ and R₉₋₁₆ are each hydrogen. In one embodiment, R₈ is —H, (C₁-C₆)-alkyl, —OR₂₅, halogen, —CF₃, —NO₂ or —CN and one of R₄ or R₅ is —H, (C₁-C₆)-alkyl, —OR₂₅, halogen, or —CF₃, and the remaining Rₐ, R_{b}, and R₁₋₇ and R₉₋₁₆ are each hydrogen. In one embodiment R₈ is —H, (C₁-C₆)-alkyl, —OR₂₅, halogen, —CF₃, —NO₂ or —CN; and Rₐ, R_{b}, and R₁₋₇ and R₉₋₁₆ are each hydrogen. In one embodiment, R₈ is —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and the remaining Rₐ, R_{b}, and R₁₋₇ and R₉₋₁₆ are each hydrogen.

In one embodiment, R₉ is —H, (C₁-C₆)-alkyl, —OR₂₅, halogen, —CF₃, —NO₂ or —CN. In another embodiment, R₉ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and the remaining Rₐ, R_{b}, and R₁₋₈ and R₁₀₋₁₆ are each hydrogen. In a further embodiment, R₉ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; one of R₁₃, R₁₄, R₁₅, and R₁₆ is —H, (C₁-C₆)-alkyl, —OR₂₅, or halogen, and the remaining Rₐ, R_{b}, and R₁₋₈ and R₁₀₋₁₆ are each hydrogen. In one embodiment, R₉ is —H, (C₁-C₆)-alkyl, —OR₂₅, halogen, —CF₃, —NO₂ or —CN and one of R₄ or R₅ is —H, (C₁-C₆)-alkyl, —OR₂₅, halogen, or —CF₃, and each the remaining Rₐ, R_{b}, and R₁₋₈ and R₁₀₋₁₆ are each hydrogen. In one embodiment R₉ is —H, (C₁-C₆)-alkyl, —OR₂₅, halogen, —CF₃, —NO₂ or —CN; and Rₐ, R_{b}, and R₁₋₈ and R₁₀₋₁₆ are each hydrogen. In one embodiment, R₉ is —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and the remaining Rₐ, R_{b}, and R₁₋₈ and R₁₀₋₁₆ are each hydrogen.

In one embodiment, R₇ is —H, (C₁-C₆)-alkyl, —OR₂₅, halogen, —CF₃, —NO₂ or —CN. In one embodiment, R₇ is —H, (C₁-C₆)-alkyl or halogen. In one embodiment, R₇ is (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and the remaining R₁₋₈ and R₁₀₋₁₆ are each hydrogen. In one embodiment, R₇ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and the remaining Rₐ, R_{b}, and R₁₋₆ and R₈₋₁₆ are each hydrogen.

In one embodiment, R₁₀ is —H, (C₁-C₆)-alkyl, —OR₂₅, halogen, —CF₃, —NO₂ or —CN.

In one embodiment, R₁₀ is —H, (C₁-C₆)-alkyl, —OR₂₅, halogen, or —CF₃. In one embodiment, R₁₀ is —H, —CH₃, —OCH₃, —F or —CF₃. In one embodiment, R₁₀ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and the remaining R₁₋₉ and R₁₁₋₁₆ are each hydrogen. In one embodiment, R₁₀ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and the remaining Rₐ, R_{b}, and R₁₋₉ and R₁₋₁₆ are each hydrogen.

In one embodiment, R₁, is —H, (C₁-C₆)-alkyl, —OR₂₅, halogen, —CF₃, —NO₂ or —CN. In one embodiment, R₁, is —H, (C₁-C₆)-alkyl, halogen, or —CF₃. In one embodiment, R₁, is —CH₃, —F or —CF₃. In one embodiment, R₁₁ is —H, (C₁-C₆)-alkyl, —OR₂₅, halogen, —CF₃, or —NO₂. In one embodiment, R₁, is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and the remaining R₁₋₁₀ and R₁₂₋₁₆ are each hydrogen. In one embodiment, R₁₁ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and the remaining Rₐ, R_{b}, R₁₋₁₀ and R₁₂₋₁₆ are each hydrogen.

In one embodiment, R₁₂ is —H, (C₁-C₆)-alkyl, —OR₂₅, halogen, —CF₃, —NO₂ or —CN. In one embodiment, R₁₂ is —H, (C₁-C₆)-alkyl, halogen, or —CF₃. In one embodiment, R₁₂ is —CH₃, —F or —CF₃. In one embodiment, R₁₂ is —H, (C₁-C₆)-alkyl, —OR₂₅, halogen, —CF₃, or —NO₂. In one embodiment, R₁₂ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and Rₐ and R_{b} are each independently —H or —CH₃; and R₁₋₁₁ and R₁₃₋₁₆ are each hydrogen. In one embodiment, R₁₂ is —H, (C₁-C₆)-alkyl, —OR₂₅, halogen, —CF₃, —NO₂ or —CN; one of R₁, R₂, R₃, R₄, R₅ and R₆ is —H, (C₁-C₆)-alkyl, —OR₂₅, halogen, or —CF₃; and the remaining Rₐ, R_{b}, R₁₋₁₁ and R₁₃₋₁₆ are each hydrogen.

In one embodiment, R₅ is —H, (C₁-C₆)-alkyl, —OR₂₅, halogen, or —CF₃ and one of R₇, R₈, R₉, R₁₀, R₁₁ and R₁₂ is —H, (C₁-C₆)-alkyl, —OR₂₅, halogen, —CF₃, —NO₂ or —CN. In another embodiment, R₅ is —H, (C₁-C₆)-alkyl, —OR₂₅, halogen, or —CF₃ and one of R₇, R₈, R₉, R₁₀, R₁₁, R₁₂ is —H, (C₁-C₆)-alkyl, —OR₂₅, halogen, —CF₃, —NO₂ or —CN; and the remaining Rₐ, R_{b}, R₁₋₄ and R₆₋₁₆ are each hydrogen. In some embodiments, R₅ is —H, (C₁-C₆)-alkyl, —OR₂₅, halogen, or —CF₃ and R₉ is —H, (C₁-C₆)-alkyl, —OR₂₅, halogen, —CF₃, —NO₂ or —CN; and the remaining Rₐ, R_{b}, R₁₋₄, R₆₋₈ and R₁₀₋₁₆ are each hydrogen. In one embodiment, R₅ is —OR₂₅ and one of R₇, R₈, R₉, R₁₀, R₁₁ and R₁₂ is —H, (C₁-C₆)-alkyl, —OR₂₅, halogen, —CF₃, —NO₂ or —CN. In one embodiment, R₅ is —OR₂₅ and R₉ is halogen. In one embodiment, R₅ is —OR₂₅ and R₉ is halogen and the remaining Rₐ, R_{b}, R₁₋₄ and R₆₋₈, R₁₀₋₁₆ are each hydrogen.

In a further embodiment, R₅ is —H, (C₁-C₆)-alkyl, —OR₂₅, halogen, or —CF₃; one of R₇, R₈, R₉, R₁₀, R₁₁, and R₁₂ is —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and the remaining Rₐ, R_{b}, R₁₋₄ and R₆₋₁₆ are each hydrogen.

In a further embodiment, R₅ is —H, (C₁-C₆)-alkyl, —OR₂₅, halogen, or —CF₃; two of R₇, R₈, R₉, R₁₀, R₁₁, and R₁₂ are each independently —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and the remaining Rₐ, R_{b}, R₁₋₄ and R₆₋₁₆ are each hydrogen.

In a further embodiment, R₅ is —H, (C₁-C₆)-alkyl, —OR₂₅, halogen, or —CF₃; three of R₇, R₈, R₉, R₁₀, R₁₁, and R₁₂ are each independently —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and the remaining Rₐ, R_{b}, R₁₋₄ and R₆₋₁₆ are each hydrogen.

In one embodiment, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; and two of R₁₀, R₁₁ and R₁₂ are each independently —H, (C₁-C₆)-alkyl, —OR₂₅, halogen, —CF₃, —NO₂ or —CN. In another embodiment, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; two of R₁₀, R₁₁, R₁₂ are each independently —H, (C₁-C₆)-alkyl, —OR₂₅, halogen, —CF₃, —NO₂ or —CN; and the remaining Rₐ, R_{b}, R₁₋₄ and R₆₋₈, R₁₀₋₁₆ are each hydrogen. In some embodiments, R₅ is —OR₂₅; R₉ is halogen; two of R₁₀, R₁₁, and R₁₂ are each independently —H, (C₁-C₆)-alkyl, —OR₂₅, halogen, —CF₃, —NO₂ or —CN; and the remaining Rₐ, R_{b}, R₁₋₄ and R₆₋₁₆ are each hydrogen. In some embodiments, R₅ is —OCH₃; R₉ is halogen; two of R₁₀, R₁₁, and R₁₂ are each independently —H, (C₁-C₆)-alkyl, —OR₂₅, halogen, —CF₃, —NO₂ or —CN; and the remaining Rₐ, R_{b}, R₁₋₄ and R₆₋₈, R₁₀₋₁₆ are each hydrogen.

In some embodiments, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; R₁₀ and R₁₂ are each independently —H, (C₁-C₆)-alkyl, —OR₂₅, halogen, —CF₃, —NO₂ or —CN; and the remaining Rₐ, R_{b}, R₁₋₄ and R₆₋₈, R₁₁, and R₁₃₋₁₆ are each hydrogen. In some embodiments, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; R₁₀ and R₁₁ are each independently —H, (C₁-C₆)-alkyl, —OR₂₅, halogen, —CF₃, —NO₂ or —CN; and the remaining Rₐ, R_{b}, R₁₋₄ and R₆₋₈, and R₁₂₋₁₆ are each hydrogen. In some embodiments, R₅ is —H, (C₁-C₆)-alkyl, —OR₂₅, halogen, or —CF₃; R₉ is —H, (C₁-C₆)-alkyl, —OR₂₅, halogen, —CF₃, —NO₂ or —CN; R₁₁ and R₁₂ are each independently —H, (C₁-C₆)-alkyl, —OR₂₅, halogen, —CF₃, —NO₂ or —CN; and the remaining Rₐ, R_{b}, R₁₋₄ and R₆₋₈, R₁₀, and R₁₃₋₁₆ are each hydrogen.

In some embodiments, R₅ is —H or —OR₂₅, R₉ is —H or halogen, R₁₀ and R₁₂ are each independently, —H, halogen, or —CF₃; and the remaining Rₐ, R_{b}, R₁₋₄ and R₆₋₈, R₁₁, and R₁₃₋₁₆ are each hydrogen.

In one embodiment, R₄ is —H, (C₁-C₆)-alkyl, —OR₂₅, halogen, or —CF₃ and one of R₇, R₈, R₉, R₁₀, R₁₁ and R₁₂ is —H, (C₁-C₆)-alkyl, —OR₂₅, halogen, —CF₃, —NO₂ or —CN. In another embodiment, R₄ is —H, (C₁-C₆)-alkyl, —OR₂₅, halogen, or —CF₃; one of R₇, R₈, R₉, R₁₀, R₁₁, and R₁₂ is —H, (C₁-C₆)-alkyl, —OR₂₅, halogen, —CF₃, —NO₂ or —CN; and the remaining Rₐ, R_{b}, R₁₋₃ and R₅₋₁₆ are each hydrogen. In a further embodiment, R₄ is —H, (C₁-C₆)-alkyl, —OR₂₅, halogen, or —CF₃; one of R₇, R₈, R₉, R₁₀, R₁₁; R₁₂ is —H, (C₁-C₆)-alkyl, —OR₂₅, halogen, —CF₃, —NO₂ or —CN; one of R₁₃, R₁₄, R₁₅, and R₁₆ is —H, (C₁-C₆)-alkyl, —OR₂₅, or halogen, and the remaining Rₐ, R_{b}, R₁₋₃ and R₅₋₁₆ are each hydrogen.

In one embodiment, one of R₁₃, R₁₄, R₁₅, and R₁₆ is —H, (C₁-C₆)-alkyl, halogen, —CF₃, or —OR₂₅.

In one embodiment, R₁, R₂, R₃, R₆, R₇, R₈, R₉, R₁₀, R₁₁, R₁₂, R₁₃, R₁₄, R₁₅, and R₁₆ are each hydrogen.

In one embodiment, R₁, R₂, R₃, R₄, R₇, R₉, R₁₀, R₁₁, and R₁₂ are each hydrogen.

In one embodiment, R₁, R₂, R₃, R₄, R₇, R₈, R₁₀, R₁₁, and R₁₂ are each hydrogen.

In one embodiment, R₁, R₂, R₃, R₄, R₇, R₈, R₉, R₁₁, and R₁₂ are each hydrogen.

In one embodiment, R₁, R₂, R₃, R₄, R₇, R₈, R₉, R₁₀, and R₁₂ are each hydrogen.

In one embodiment, R₁, R₂, R₃, R₄, R₇, R₈, R₉, R₁₀, and R₁₁ are each hydrogen.

In one embodiment, R₁, R₂, R₃, R₄, R₇, R₈, and R₁₁ are each hydrogen.

In one embodiment, R₁, R₂, R₃, R₄, R₇, R₈, R₉ and R₁₁ are each hydrogen.

In one embodiment, R₁, R₂, R₃, R₄, R₅, R₆, R₇, R₈, R₉, and R₁₂ are each hydrogen.

In another embodiment, R₁₃, R₁₄, R₁₅, and R₁₆ are each hydrogen.

In one embodiment, R₃, R₆, R₇, R₈, R₉, R₁₂, R₁₃, R₁₄, R₁₅, and R₁₆ are each hydrogen.

In one embodiment, R₁, R₂, R₃, R₄, R₆, R₇, R₈, and R₁₁ are each hydrogen.

In one embodiment, R₁, R₂, R₃, R₄, R₆, R₇, R₈, R₁₁, R₁₃, R₁₄, R₁₅, and R₁₆ are each hydrogen.

In one embodiment, R₁, R₂, R₃, R₄, R₆, R₇, R₈, R₁₀, R₁₁, R₁₂, R₁₃, R₁₄, R₁₅, and R₁₆ are each hydrogen.

In one embodiment, R₁, R₂, R₃, R₄, R₆, R₇, R₈, R₉, R₁₀, R₁₁ and R₁₂ are each hydrogen.

In one embodiment, R₁, R₂, R₃, R₄, R₆, R₇, R₈, R₉, R₁₀, R₁₁, R₁₂ R₁₃, R₁₄, R₁₅, and R₁₆ are each hydrogen.

In one embodiment, R₁ is —H, —CF₃ or (C₁-C₆)-alkyl; R₄ and R₅ are each —H, halogen, —OR₂₅, or —CF₃; R₇, R₈, R₉, R₁₀, R₁₁, and R₁₂ are each —H, halogen, -alkyl, —OR₂₅, —CF₃, or —NO₂; and R₁₆ is —H or —CH₃.

In one embodiment, any one of R₁, R₂, R₃, R₄, R₅, and R₆ is —H, (C₁-C₆)-alkyl, —OR₂₅, halogen or —CF₃; and any one of R₇, R₈, R₉, R₁₀, R₁₁, and R₁₂ is —H, (C₁-C₆)-alkyl, —OR₂₅, halogen, or —CF₃, —NO₂, or —CN.

In one embodiment, any one of R₁, R₂, R₃, R₄, R₅, and R₆ is —H, (C₁-C₆)-alkyl, —OR₂₅, halogen, —CF₃, —NO₂, or —CN; and any two of R₇, R₈, R₉, R₁₀, R₁₁, and R₁₂ are each independently —H, (C₁-C₆)-alkyl, —OR₂₅, halogen, —CF₃.

In one embodiment, any one of R₁, R₂, R₃, R₄, R₅, and R₆ is —H, (C₁-C₆)-alkyl, —OR₂₅, halogen, —CF₃, —NO₂, or —CN; and any three of R₇, R₈, R₉, R₁₀, R₁₁, and R₁₂ are each independently —H, (C₁-C₆)-alkyl, —OR₂₅, halogen, —CF₃.

In one embodiment, any one of R₁, R₂, R₃, R₄, R₅, and R₆ is —H, (C₁-C₆)-alkyl, —OR₂₅, halogen or —CF₃; and any one of R₁₃, R₁₄, R₁₅, and R₁₆ is —H, (C₁-C₆)-alkyl, —OR₂₅, halogen or —CF₃.

In one embodiment, any one of R₇, R₈, R₉, R₁₀, R₁₁, and R₁₂ is —H, C₁-C₆)-alkyl, —OR₂₅, halogen, —CF₃, —NO₂, or —CN; and any one of R₁₃, R₁₄, R₁₅, and R₁₆ is —H, (C₁-C₆)-alkyl, —OR₂₅, halogen, —CF₃.

In one embodiment, R₄ is —H, (C₁-C₆)-alkyl, —OR₂₅, halogen, or —CF₃ and any one of R₁₃, R₁₄, R₁₅, and R₁₆ is —H, (C₁-C₆)-alkyl, —OR₂₅, halogen or —CF₃; and any one of R₇, R₈, R₉, R₁₀, R₁₁, and R₁₂ is —H, (C₁-C₆)-alkyl, —OR₂₅, halogen, or —CF₃, —NO₂, or —CN.

In one embodiment, R₄ is —H, (C₁-C₆)-alkyl, —OR₂₅, halogen, or —CF₃ and any one of R₁₃, R₁₄, R₁₅, and R₁₆ is —H, (C₁-C₆)-alkyl, —OR₂₅, halogen or —CF₃; and any two of R₇, R₈, R₉, R₁₀, R₁₁, and R₁₂ are each independently —H, (C₁-C₆)-alkyl, —OR₂₅, halogen, or —CF₃, —NO₂, or —CN; wherein the any two of R₇, R₈, R₉, R₁₀, R₁₁, and R₁₂ can be either on the same ring of the quinoline or on different rings.

In one embodiment, R₅ is —H, (C₁-C₆)-alkyl, —OR₂₅, halogen, or —CF₃ and any one of R₁₃, R₁₄, R₁₅, and R₁₆ is —H, (C₁-C₆)-alkyl, —OR₂₅, halogen or —CF₃; and any one of R₇, R₈, R₉, R₁₀, R₁₁, and R₁₂ is —H, (C₁-C₆)-alkyl, —OR₂₅, halogen, or —CF₃, —NO₂, or —CN. In one embodiment, R₅ is —OR₂₅; any one of R₁₃, R₁₄, R₁₅, and R₁₆ is —H, (C₁-C₆)-alkyl, —OR₂₅, halogen or —CF₃; and any one of R₇, R₈, R₉, R₁₀, R₁₁, and R₁₂ is —H, (C₁-C₆)-alkyl, —OR₂₅, halogen, or —CF₃, —NO₂, or —CN.

In one embodiment, R₅ is —H, (C₁-C₆)-alkyl, —OR₂₅, halogen, or —CF₃ and any one of R₁₃, R₁₄, R₁₅, and R₁₆ is —H, (C₁-C₆)-alkyl, —OR₂₅, halogen or —CF₃; and any two of R₇, R₈, R₉, R₁₀, R₁₁, and R₁₂ are each independently —H, (C₁-C₆)-alkyl, —OR₂₅, halogen, or —CF₃, —NO₂, or —CN; wherein the any two of R₇, R₈, R₉, R₁₀, R₁₁, and R₁₂ can be either on the same ring of the quinoline or on different rings.

In one embodiment, R₅ is —OR₂₅; R₉ is halogen; any one of R₁₃, R₁₄, R₁₅, and R₁₆ is —H, (C₁-C₆)-alkyl, —OR₂₅, halogen or —CF₃; and any two of R₇, R₈, R₁₀, R₁₁, and R₁₂ are each independently —OR₂₅, halogen, or —CF₃; wherein the any two of R₇, R₈, R₉, R₁₀, R₁₁, and R₁₂ can be either on the same ring of the quinoline or on different rings.

In one embodiment, R₁ is —H or (C₁-C₆)-alkyl; R₂, R₈, and R₉ are each —H or halogen; R₄ is —H, halogen, —OR₂₅, or —CF₃; R₅ is —H, halogen, or —OR₂₅; and R₃, R₆, R₇, R₁₂, R₁₃, R₁₄, R₁₅, R₁₆, Rₐ and R_{b} are each hydrogen.

In one embodiment, R₁ is —H or —CH₃; R₂, R₈, and R₉ are each —H or F; R₄ is —H, F, —OCH₃, or —CF₃; R₅ is —H, F, or —OCH₃; and R₃, R₆, R₇, R₁₀, R₁₁, R₁₂, R₁₃, R₁₄, R₁₅, R₁₆, Rₐ and R_{b} are each hydrogen.

In one embodiment, R₂₅ is (C₁-C₆)-haloalkyl.

In another embodiment, R₂₅ is (C₁-C₆)-fluoroalkyl.

In one embodiment, R₂₅ is (C₁-C₆)-alkyl. In one embodiment, R₂₅ is —CH₃.

In some embodiments, the compound of formula (I) is selected from the group consisting of:
6-methoxy-8-[4-(1-quinolin-8-yl-piperidin-4-yl)-piperazin-1-yl]-quinoline;
6-fluoro-8-[4-(1-quinolin-8-yl-piperidin-4-yl)-piperazin-1-yl]-quinoline;
5-fluoro-8-[4-(1-quinolin-8-yl-piperidin-4-yl)-piperazin-1-yl]-quinoline;
7-fluoro-8-(4-(4-(6-methoxyquinolin-8-yl)piperazin-1-yl)piperidin-1-yl)quinoline;
6-fluoro-8-{4-[1-(8-fluoroquinolin-7-yl)piperidin-4-yl]piperazin-1-yl}quinoline;
3-trifluoromethyl-8-(4-(4-(6-methoxyquinolin-8-yl)piperazin-1-yl)piperidin-1-yl)quinoline;
6-methoxy-8-(4-(1-(quinolin-8-ylmethyl)piperidin-4-yl)piperazin-1-yl)quinoline;
5-fluoro-4-methoxy-8-(4-(4-(6-methoxyquinolin-8-yl)piperazin-1-yl)piperidin-1-yl)-2-(trifluoromethyl)quinoline;
5-fluoro-8-(4-(4-(6-methoxyquinolin-8-yl)piperazin-1-yl)piperidin-1-yl)quinoline;
8-[4-(1-quinolin-8-yl-piperidin-4-yl)-piperazin-1-yl]-quinoline;
6-chloro-8-[4-(4-(6-chloro)-quinolin-8-yl-piperidin-1-yl)-piperazin-1-yl]-quinoline;
6-fluoro-8-[4-(4-(6-chloro)-quinolin-8-yl-piperidin-1-yl)-piperazin-1-yl]-quinoline;
5-chloro-8-[4-(1-quinolin-8-yl-piperidin-4-yl)-piperazin-1-yl]-quinoline;
2-methyl-8-[4-(1-quinolin-8-yl-piperidin-4-yl)-piperazin-1-yl]-quinoline;
6-chloro-8-[4-(1-quinolin-8-yl-piperidin-4-yl)-piperazin-1-yl]-quinoline;
8-[4-(1-quinolin-8-yl-piperidin-4-yl)-piperazin-1-yl]-5-trifluoromethyl-quinoline;
5-methoxy-8-[4-(1-quinolin-8-yl-piperidin-4-yl)-piperazin-1-yl]-quinoline;
5-fluoro-8-[4-(4-quinolin-8-yl-piperazin-1-yl)-piperidin-1-yl]-quinoline;
6-methoxy-8-[4-(2-methylquinolin-8-yl-piperidin-4-yl)-piperazin-1-yl]-quinoline;
6-fluoro-8-(4-(1-(2-methylquinolin-8-yl)piperidin-4-yl)piperazin-1-yl)quinoline;
6-methoxy-8-[4-(3-methylquinolin-8-yl-piperidin-4-yl)-piperazin-1-yl]-quinoline;
6-methoxy-8-(4-(1-(4-methylquinolin-8-yl)piperidin-4-yl)piperazin-1-yl)quinoline;
6-methoxy-8-(4-(1-(2,4-dimethylquinolin-8-yl)piperidin-4-yl)piperazin-1-yl)quinoline;
6-methoxy-8-(4-(1-(2,4-dimethyl-5-fluoroquinolin-8-yl)piperidin-4-yl)piperazin-1-yl)quinoline;
6-methoxy-8-(4-(1-(2-(trifluoromethyl)quinolin-8-yl)piperidin-4-yl)piperazin-1-yl)quinoline;
6-fluoro-8-(4-(1-(5-fluoroquinolin-8-yl)piperidin-4-yl)piperazin-1-yl)quinoline;
6-methoxy-8-(4-(1-(6-bromoquinolin-8-yl)piperidin-4-yl)piperazin-1-yl)quinoline;
6-methoxy-8-(4-(1-(6-fluoroquinolin-8-yl)piperidin-4-yl)piperazin-1-yl)quinoline;
6-fluoro-8-(4-(1-(7-fluoroquinolin-8-yl)piperidin-4-yl)piperazin-1-yl)quinoline;
6-methoxy-8-{4-[1-(8-fluoroquinolin-7-yl)piperidin-4-yl]piperazin-1-yl}quinoline;
6-methoxy-8-{4-[1-(2-trifluoromethyl-4-methoxyquinolin-7-yl)piperidin-4-yl]piperazin-1-yl}quinoline;
6-methoxy-8-(4-(1-(2-trifluoromethyl-4-methoxyquinolin-8-yl)piperidin-4-yl)piperazin-1-yl)quinoline;
5-fluoro-8-(4-(4-(6-methoxyquinolin-8-yl)piperazin-1-yl)piperidin-1-yl)-2-trifluoromethylquinoline;
5-fluoro-8-(4-(4-(6-methoxyquinolin-8-yl)piperazin-1-yl)piperidin-1-yl)-3-trifluoromethylquinoline;
5-fluoro-8-(4-(4-(6-methoxyquinolin-8-yl)piperazin-1-yl)piperidin-1-yl)-4-trifluoromethylquinoline;
2,5-difluoro-8-(4-(4-(6-methoxyquinolin-8-yl)piperazin-1-yl)piperidin-1-yl)quinoline;
3,5-difluoro-8-(4-(4-(6-methoxyquinolin-8-yl)piperazin-1-yl)piperidin-1-yl)quinoline;
4,5-difluoro-8-(4-(4-(6-methoxyquinolin-8-yl)piperazin-1-yl)piperidin-1-yl)quinoline;
   and pharmaceutically acceptable salts thereof.

In one embodiment, the compound of formula (I) is 5-fluoro-8-{4-[4-(6-methoxyquinolin-8-yl)piperazin-1-yl]piperidin-1-yl}quinoline or a pharmaceutically acceptable salt thereof. In one embodiment, the compound of formula (I) is 5-fluoro-8-{4-[4-(6-methoxyquinolin-8-yl)piperazin-1-yl]piperidin-1-yl}quinoline succinate. In one embodiment, the compound of formula (I) is 5-fluoro-8-{4-[4-(6-methoxyquinolin-8-yl)piperazin-1-yl]piperidin-1-yl}quinoline trisuccinate.

The compounds and pharmaceutically acceptable salts of compounds described herein can contain an asymmetric carbon atom and some of the compounds or pharmaceutically acceptable salts of compounds can contain one or more asymmetric centers, and can thus give rise to optical isomers and diastereomers. While described without respect to stereochemistry herein, the present invention includes such optical isomers and diastereomers, as well as racemic and resolved, enantiomerically pure R and S stereoisomers, and also other mixtures of the R and S stereoisomers and pharmaceutically acceptable salts thereof. Where a stereoisomer is preferred, it can in some embodiments be provided substantially free of its corresponding opposite enantiomer.

In addition, the compounds and pharmaceutically acceptable salts of compounds described herein can exist as polymorphs. Such polymorphs can be transient or isolatable as a stable product. Examples of some polymorphs of the compounds described herein are included in U.S. Provisional Patent Publication No. 2007/0299083A1, entitled “6-Methoxy-8-[4-(1-(5-Fluoro)-Quinolin-8-yl-Piperidin-4-yl)-piperazin-1-yl]-Quinoline Hydrochloric Acid Salts”, filed Jun. 8, 2007, and U.S. patent application Ser. No. 11/811,022, entitled “Succinate Salts Of 6-Methoxy-8-[4-(1-(5-Fluoro)-Quinolin-8-yl-Piperidin-4-yl)-piperazin-1-yl]-Quinoline And Crystalline Forms Thereof”, filed Jun. 8, 2007. For example, in some embodiments, the compounds or pharmaceutically acceptable salts of the compounds described herein are Forms A, B, C, or D of 6-methoxy-8-[4-(1-(5-fluoro)-quinolin-8-yl-piperidin-4-yl)-piperazin-1-yl]-quinoline trisuccinate.

Prodrugs of the compounds or pharmaceutically acceptable salts of compounds are also within the scope of the present invention.

The compounds described herein are prepared according to methods described U.S. Patent Publication No. 2007/0027160 and U.S. Patent Publication No. 2007/146072 filed Jun. 7, 2007, entitled “Process For Synthesizing piperazine-Piperidine Compounds”.

In some embodiments, the sustained-release tablet formulations degrade so that the Active Pharmaceutical Ingredient (API) (i.e., compound of formula (I)) is released to provide a Cₘₐₓ of the API between about 0.5 and about 18 hours after administration. In some embodiments, the formulations degrade so that the compound of formula (I) is released to provide a Cₘₐₓ of the API between about 2 and about 16 hours after administration. In some embodiments, the formulations degrade so that the compound of formula (I) is released to provide a Cₘₐₓ of the API between about 4 and about 12 hours after administration.

In certain embodiments, the sustained-release tablet formulations described herein include:

(a) a compound of formula (I), or a pharmaceutically acceptable salt thereof, as described hereinabove;

(b) at least one organic acid;

(c) at least one release controller;

(d) at least one filler; and

(e) at least one lubricant,

characterized in that the formulation releases at least 45% by weight of the compound of formula (I) or a pharmaceutically acceptable salt thereof, after 8 hours in a dissolution test apparatus having a rotation speed of 75 rpm, and a single stage dissolution medium containing pH 4.5 buffer at 37° C. Dissolution test apparatuses are well known to those of skill in the art. In one embodiment, the dissolution test apparatus is a basket apparatus or a paddle apparatus. In one embodiment, the dissolution test apparatus is a paddle apparatus. A nonlimiting example of a paddle apparatus is Apparatus 2 described in the United States Pharmacopoeia (USP29-NF24, page 2673). In this apparatus, a paddle formed from a blade and shaft is used as the stirring element in the dissolution test. The shaft is positioned so the axis is not more than 2 mm from the vertical axis of the vessel at any point and rotates smoothly without significant wobble that could affect the results. The distance between the bottom of the blade of the paddle and the bottom of the test vessel is 25±2 mm.

In certain embodiments, the formulation releases at least 15% by weight of the compound of formula (I) or a pharmaceutically acceptable salt thereof after 2 hours under the one stage dissolution condition as described above. In certain other embodiments, the formulation releases less than 40% by weight, or less than 20% by weight of the compound of formula (I) or a pharmaceutically acceptable salt thereof after 2 hours under the one stage dissolution condition.

In certain embodiments, the formulation releases less than 60% by weight, or less than 40% by weight of the compound of formula (I) or a pharmaceutically acceptable salt thereof after 4 hours under the one stage dissolution condition. In some cases, less than 70% by weight, or less than 50% by weight of the compound of formula (I) or a pharmaceutically acceptable salt thereof, is released after 6 hours under the one stage dissolution condition as described above.

In certain embodiments, the sustained-release tablet formulations described herein include:

(a) a compound of formula (I), or a pharmaceutically acceptable salt thereof, as described hereinabove;

(b) at least one organic acid;

(c) at least one release controller;

(d) at least one filler; and

(e) at least one lubricant,

characterized in that the formulation releases at least 60% by weight of the compound of formula (I) or a pharmaceutically acceptable salt thereof, after 8 hours in a dissolution test apparatus having a rotation speed of 75 rpm, and a two-stage dissolution medium containing (i) pH 1 buffer at 37° C. for two hours, and followed by (ii) pH 6.5 buffer with 1% SLS for additional 6 hours at 37° C. As discussed above, dissolution test apparatuses are known to those of skill in the art. In one embodiment, the dissolution test apparatus is a basket apparatus or a paddle apparatus. In one embodiment, the dissolution test apparatus is a paddle apparatus. A nonlimiting example of a paddle apparatus is Apparatus 2 described in the United States Pharmacopoeia (USP29-NF24, page 2673), described herein.

In certain embodiments, the formulation releases at least 35% by weight of the compound of formula (I) or a pharmaceutically acceptable salt thereof after 2 hours under the two-stage dissolution condition as described above. In certain other embodiments, the formulation releases less than 55% by weight, or less than 40% by weight of the compound of formula (I) or a pharmaceutically acceptable salt thereof after 2 hours under the two-stage dissolution condition.

In certain embodiments, the formulation releases less than 70% by weight, or less than 50% by weight of the compound of formula (I) or a pharmaceutically acceptable salt thereof after 4 hours under the two-stage dissolution condition. In some cases, less than 80% by weight, or less than 60% by weight of the compound of formula (I) or a pharmaceutically acceptable salt thereof, is released after 6 hours under the two-stage dissolution condition as described above.

In certain embodiments, the organic acid is citric acid, ascorbic acid, aspartic acid, glutamic acid, tartaric acid, succinic acid, malic acid, erythorbic acid, propionic acid, lactic acid, oleic acid, fumaric acid, benzoic acid, or alginic acid. In certain embodiments, the organic acid is citric acid or succinic acid. In certain embodiments, the organic acid makes up about 2% to about 20% by weight of the formulation, for example, at least about 5%, at least about 8%, at least about 10%, at least about 12%, at least about 15%, at least about 18%. In certain other embodiments, the organic acid makes up about 5% to about 15% by weight of the formulation or from about 8% to about 10% by weight of the formulation.

The sustained-release controller, or release controller, typically, although not necessarily, includes a polymeric material as the sustained-release component. Such materials include any polymer material suitable for pharmaceutical dosage forms that retard the release of drug substances from such dosage forms. Examples of suitable polymers for use as the sustained-release coat or release controller can be found in Remington's Pharmaceutical Sciences, 18th Ed., Gennaro, ed., Mack Publishing Co., Easton, Pa., 1990. In some embodiments, the sustained-release component, or release controller, includes, without limitation, one or more of ethylcelluloses, polyvinyl acetates, polymers or copolymers or acrylates or methacrylates, or cellulose acetate. In some embodiments, the sustained-release component, or release controller, includes, without limitation, one or more of polymethacrylates, methacrylic acid-methacrylic acid ester copolymers, acrylate methacrylate copolymers, ethylacrylate/methylmethacrylate copolymers, cellulose acetate, ethylcellulose, hydroxypropyl methyl cellulose, high viscosity matrix forming hydroxypropyl methyl celluloses such as Methocel® K4M, Methocel® K15M, Methocel® K100M, Methocel® E4M, and low viscosity matrix forming hydroxypropyl methyl celluloses such as Methocel® K100LV, Methocel® E50LV, Methocel® E5, Methocel® E15LV. In some embodiments, the sustained-release component, or release controller, includes, without limitation, hydroxypropyl methyl cellulose, high viscosity matrix forming hydroxypropyl methyl celluloses, and low viscosity matrix forming hydroxypropyl methyl celluloses. In some embodiments, the sustained-release coat includes an ethylcellulose-based product, such as the commercially available Surelease™ aqueous ethylcellulose dispersion product (Colorcon, Inc., West Point, Pa.). For example, in some embodiments, the sustained-release coat includes Surelease-E-7-19010, containing ethylcellulose and other ingredients including ammonium hydroxide. Combinations of different coating materials may also be used as a sustained-release coating. The sustained-release coat or component initially can form a premix, solution, or suspension of any of the above coating materials.

In certain other embodiments, the release controller makes up about 10% to about 60% by weight of the formulation, for example, at least about 15%, at least about 20%, at least about 25%, at least about 30%, at least about 35%, at least about 40%, at least about 45%, at least about 50%, about 55%. In some embodiments, the release controller makes up about 30% to about 50% by weight of the formulation. In some embodiments, the release controller makes up about 20% to about 50% by weight of the formulation.

In certain embodiments, the filler is microcrystalline cellulose, silicified microcrystalline cellulose, lactose, calcium carbonate, calcium sulfate, calcium phosphate, sodium chloride, maltodextrin, dextrose, fructose, maltose, mannitol, starch, sucrose, or kaolin. In certain embodiments, the filler is microcrystalline cellulose, silicified microcrystalline cellulose, lactose, calcium carbonate, calcium sulfate, or calcium phosphate. In certain embodiments, the filler is microcrystalline cellulose or silicified microcrystalline cellulose. In certain other embodiments, the filler makes up about 25% to about 65% by weight of the formulation, for example, at least about 30%, at least about 35%, at least about 40%, at least about 45%, at least about 50%, at least about 55%, or at least about 60%. In some embodiments, the filler makes up about 35% to about 65% by weight of the formulation

In certain other embodiments, the lubricant is magnesium stearate, calcium stearate, stearic acid, talc, hydrogenated vegetable oils, polyethylene glycol, or colloidal silicon dioxide. In some embodiments, the lubricant is magnesium stearate, calcium stearate, or stearic acid. In certain other embodiments, the lubricant makes up about 0.1% to about 5% by weight of the formulation, for example, at least about 0.2%, at least about 0.3%, at least about 0.4%, at least about 0.5%, at least about 1%, at least about 2%, at least about 3%, and at least about 4%. In some embodiments, the lubricant makes up about 0.1% to about 1% by weight of the formulation

In certain embodiments, the sustained-release tablet formulation contains from about 0.1 mg to about 100 mg of the compound of formula (I), or a pharmaceutically acceptable salt thereof. In some cases, the formulation contains from about 0.5 mg to about 25 mg of the compound of formula (I), or a pharmaceutically acceptable salt thereof, for example, about 2.5 mg, about 5.0 mg, and about 10.0 mg, about 15.0 mg, about 20.0 mg. In certain other embodiments, the compound of formula (I), or a pharmaceutically acceptable salt thereof, makes up about 0.02% to about 20% by weight of the formulation, expressed in the form of its free base, for example, about 0.2%, about 1%, about 4%, about 5%, about 10%, and about 15%. In some embodiments, the compound of formula (I), or a pharmaceutically acceptable salt thereof, makes up about 0.1% to about 1% by weight of the formulation, about 0.1% to about 5%, about 1% to about 5%, about 1% to about 10%, about 1% to about 15%, about 5% to about 15%, about 5% to about 15%.

The amount of the API (active pharmaceutical ingredient; i.e., compound of formula (I)) in the oral unit dosage form, with as a single or multiple dosage, is an amount that is effective for treating or preventing a 5-HT_{1A}-related disorder. As one of skill in the art will recognize, the precise dose to be employed will depend on a variety of factors, examples of which include the condition itself, the seriousness of the condition being treated, the particular formulation used, as well as various physical factors related to the individual being treated. In vitro or in vivo assays can optionally be employed to help identify optimal dosage ranges. To determine the effective amount of the formulation being administered, a physician may, for example, evaluate the effects of a given formulation of a compound of formula (I) in the patient by incrementally increasing the dosage until the desired symptomatic relief level is achieved. The dose regimen may then be further modified to achieve the desired result. For oral administration, in some embodiments, the formulations described herein are incrementally increased in a patient in an amount from about 0.001 mg/kg to about 10 mg/kg until the desired symptomatic relief level is achieved. In some embodiments, the patient is administered the formulations described herein as a single oral dose (e.g., one 10 mg tablet) or as a multiple oral dose (e.g., three 3 mg tablets; two 5 mg tablets; four 2.5 mg tablets).

In some embodiments, however, a dosage (whether in unit or multiple dosage form) for daily oral administration will range from about range from about 0.001 mg to about 600 mg per day, in one embodiment, from about 1 mg to about 600 mg per day, in another embodiment, from about 10 mg to about 400 mg per day, in another embodiment, from about 10 mg to about 200 mg per day, in another embodiment, from about 10 mg to about 100 mg per day, in another embodiment, from about 1 mg to about 100 mg per day, in another embodiment, from about 1 mg to about 50 mg per day, in another embodiment, from about 1 mg to about 25 mg per day, and in another embodiment, from about 1 mg to about 10 mg per day. In one embodiment, the dosage for daily oral administration will be 2.5 mg per day, 5 mg per day, 7.5 mg per day, 10 mg per day, 15 mg per day, 20 mg per day, 25 mg per day, 30 mg per day, 35 mg per day, 40 mg per day, 45 mg per day, or 50 mg per day.

The oral unit dosage forms described herein (tablets) generally contain from about 0.25 mg to about 500 mg of the API (i.e., compound of formula (I)). In some embodiments, the oral unit dosage forms contain from about 0.25 mg to about 400 mg of the API, or about 0.25 mg to about 300 mg of the API, or about 0.25 mg to about 250 mg of the API, or about 0.25 mg to about 200 mg of the API, or about 0.25 mg to about 100 mg of the API, or about 0.25 mg to about 75 mg of the API, or about 0.25 mg to about 50 mg of the API, or about 0.25 mg to about 25 mg of the API, or about 0.25 mg to about 15 mg of the API. In some embodiments, the oral unit dosage forms contain about 0.25 mg of the API, or about 0.5 mg of the API, or about 0.75 mg of API, or about 1 mg of the API, or about 1.5 mg of the API, or about 2 mg of the API, or about 2.5 mg of the API, or about 3 mg of the API, or about 3.5 mg of the API, or about 4 mg of the API, or about 4.5 mg of the API, or about 5 mg of the API, or about 6 mg of the API, or about 7 mg of the API, or about 8 mg of the API, or about 9 mg of the API, or about 10 mg of the API, or about 15 mg of the API, or about 20 mg of the API, or about 25 mg of the API, or about 50 mg of the API, or about 100 mg of the API.

In some embodiments, the compound of formula (I) is present in the solid pharmaceutical dosage form at a level of about 1% by weight to about 75% by weight based on the total weight of the pharmaceutical formulation. In some embodiments, the compound is present at a level of about 1% by weight to about 50% by weight, about 1% by weight to about 25% by weight, about 1% by weight to about 15% by weight, or at a level of about 1% by weight to about 10% by weight, based on the total weight of the solid pharmaceutical dosage form.

In some embodiments, the pharmaceutical formulations described herein further include one or more other pharmaceutical agents. In some embodiments, the other therapeutic agent is administered in an effective amount. In some embodiments, the one or more other pharmaceutical agents are separate from the pharmaceutical formulations described herein. In some embodiments, the one or more other pharmaceutical agents are administered simultaneously and/or successively with the pharmaceutical formulations described herein.

Effective amounts of the other therapeutic agents are known to those skilled in the art. However, it is within the skilled artisan's purview to determine the other therapeutic agent's optimal effective amount range. The compound or a pharmaceutically acceptable salt of the compound and the other therapeutic agent can act additively or, in some embodiments, synergistically. In one embodiment, where another therapeutic agent is administered with the compound of formula (I), the effective amount of the compound or a pharmaceutically acceptable salt of the compound is less than its effective amount would be where the other therapeutic agent is not administered. In this case, without being bound by theory, it is believed that the compound or a pharmaceutically acceptable salt of the compound and the other therapeutic agent act synergistically. In some cases, the patient in need of treatment is being treated with one or more other therapeutic agents. In some cases, the patient in need of treatment is being treated with at least two other therapeutic agents.

In one embodiment, the other therapeutic agent is selected from the group consisting of one or more anti-depressant agents, anti-anxiety agents, anti-psychotic agents, or cognitive enhancers. Examples of classes of antidepressants that can be used in combination with the active compounds of this invention include norepinephrine reuptake inhibitors, selective serotonin reuptake inhibitors (SSRIs), NK-1 receptor antagonists, monoamine oxidase inhibitors (MAOs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Suitable selective serotonin reuptake inhibitors include fluoxetine, citolopram, escitalopram, fluvoxamine, paroxetine and sertraline. Examples of monoamine oxidase inhibitors include isocarboxazid, phenelzine, and tranylcypromine. Suitable reversible inhibitors of monoamine oxidase include moclobemide. Suitable serotonin and noradrenaline reuptake inhibitors of use in the present invention include venlafaxine, nefazodone, milnacipran, and duloxetine. Suitable CRF antagonists include those compounds described in International Patent Publication Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in International Patent Publication WO 01/77100.

Anti-anxiety agents that can be used in combination with the active compounds of this invention include without limitation benzodiazepines and serotonin 1A (5-HT_{1A}) agonists or antagonists, especially 5-HT_{1A} partial agonists, and corticotropin releasing factor (CRF) antagonists. Exemplary suitable benzodiazepines include alprazolam, chlordiazepoxide, clonazepam, chlorazepate, diazepam, halazepam, lorazepam, oxazepam, and prazepam. Exemplary suitable 5-HT_{1A} receptor agonists or antagonists include buspirone, flesinoxan, gepirone and ipsapirone.

Anti-psychotic agents that are used in combination with the active compounds of this invention include without limitation aliphatic phenothiazine, a piperazine phenothiazine, a butyrophenone, a substituted benzamide, and a thioxanthine. Additional examples of such drugs include without limitation haloperidol, olanzapine, clozapine, risperidone, pimozide, aripiprazol, and ziprasidone. In some cases, the drug is an anticonvulsant, e.g. phenobarbital, phenyloin, primidone, or carbamazepine.

Cognitive enhancers that are co-administered with the pharmaceutical formulations described herein include, without limitation, drugs that modulate neurotransmitter levels (e.g., acetylcholinesterase or cholinesterase inhibitors, cholinergic receptor agonists or serotonin receptor antagonists), drugs that modulate the level of soluble Aβ, amyloid fibril formation, or amyloid plaque burden (e.g. γ-secretase inhibitors, β-secretase inhibitors, antibody therapies, and degradative enzymes), and drugs that protect neuronal integrity (e.g., antioxidants, kinase inhibitors, caspase inhibitors, and hormones). Other representative candidate drugs that are co-administered with the compounds of the invention include cholinesterase inhibitors, (e.g., tacrine (COGNEX®), donepezil (ARICEPT®), rivastigmine (EXELON®) galantamine (REMINYL®), metrifonate, physostigmine, and Huperzine A), N-methyl-D-aspartate (NMDA) antagonists and agonists (e.g., dextromethorphan, memantine, dizocilpine maleate (MK-801), xenon, remacemide, eliprodil, amantadine, D-cycloserine, felbamate, ifenprodil, CP-101606 (Pfizer), Delucemine, and compounds described in U.S. Pat. Nos. 6,821,985 and 6,635,270), ampakines (e.g., cyclothiazide, aniracetam, CX-516 (Ampalex®), CX-717, CX-516, CX-614, and CX-691 (Cortex Pharmaceuticals, Inc. Irvine, Calif.), 7-chloro-3-methyl-3-4-dihydro-2H-1,2,4-benzothiadiazine S,S-dioxide (see Zivkovic et al., 1995, J. Pharmacol. Exp. Therap., 272:300-309; Thompson et al., 1995, Proc. Natl. Acad. Sci. USA, 92:7667-7671), 3-bicyclo[2,2, 1]hept-5-en-2-yl-6-chloro-3,4-dihydro-2H-1,2,4-benzothiadiazine-7-sulfonamide-1,1-dioxide (Yamada, et al., 1993, J. Neurosc. 13:3904-3915); 7-fluoro-3-methyl-5-ethyl-1,2,4-benzothiadiazine-S,S-dioxide; and compounds described in U.S. Pat. No. 6,620,808 and International Patent Publication Nos. WO 94/02475, WO 96/38414, WO 97/36907, WO 99/51240, and WO 99/42456), benzodiazepine (BZD)/GABA receptor complex modulators (e.g., progabide, gengabine, zaleplon, and compounds described in U.S. Pat. Nos. 5,538,956, 5,260,331, and 5,422,355); serotonin antagonists (e.g., 5-HT receptor modulators, including other 5-HT_{1A} antagonist compounds and 5-HT₆ antagonists (including without limitation compounds described in U.S. Pat. Nos. 6,727,236, 6,825,212, 6,995,176, and 7,041,695)); nicotinics (e.g., niacin); muscarinics (e.g., xanomeline, CDD-0102, cevimeline, talsaclidine, oxybutin, tolterodine, propiverine, tropsium chloride and darifenacin); monoamine oxidase type B (MAO B) inhibitors (e.g., rasagiline, selegiline, deprenyl, lazabemide, safinamide, clorgyline, pargyline, N-(2-aminoethyl)-4-chlorobenzamide hydrochloride, and N-(2-aminoethyl)-5(3-fluorophenyl)-4-thiazolecarboxamide hydrochloride); phosphodiesterase (PDE) inhibitors (e.g., PDE IV inhibitors, roflumilast, arofylline, cilomilast, rolipram, RO-20-1724, theophylline, denbufylline, ARIFLO, CDP-840 (a tri-aryl ethane) CP80633 (a pyrimidone), RP 73401 (Rhone-Poulenc Rorer), denbufylline (SmithKline Beecham), arofylline (Almirall), CP-77,059 (Pfizer), pyrid[2,3d]pyridazin-5-ones (Syntex), EP-685479 (Bayer), T-440 (Tanabe Seiyaku), and SDZ-ISQ-844 (Novartis)); G proteins; channel modulators; immunotherapeutics (e.g., compounds described in U.S. Patent Application Publication Nos. US 2005/0197356 and US 2005/0197379); anti-amyloid or amyloid lowering agents (e.g., bapineuzumab and compounds described in U.S. Pat. No. 6,878,742 or U.S. Patent Application Publication Nos. US 2005/0282825 or US 2005/0282826); statins and peroxisome proliferators activated receptor (PPARS) modulators (e.g., gemfibrozil (LOPID®), fenofibrate (TRICOR®), rosiglitazone maleate (AVANDIA®), pioglitazone (Actos™), rosiglitazone (Avandia™), clofibrate and bezafibrate); cysteinyl protease inhibitors; an inhibitor of receptor for advanced glycation endproduct (RAGE) (e.g., aminoguanidine, pyridoxaminem carnosine, phenazinediamine, OPB-9195, and tenilsetam); direct or indirect neurotropic agents (e.g., Cerebrolysin®, piracetam, oxiracetam, AIT-082 (Emilieu, 2000, Arch. Neurol. 57:454)); beta-secretase (BACE) inhibitors, α-secretase, immunophilins, caspase-3 inhibitors, Src kinase inhibitors, tissue plasminogen activator (TPA) activators, AMPA (alpha-amino-3-hydroxy-5-methyl-4-isoxazolepropionic acid) modulators, M4 agonists, JNK3 inhibitors, LXR agonists, H3 antagonists, and angiotensin IV antagonists. Other cognition enhancers include, without limitation, acetyl-1-carnitine, citicholine, huperzine, DMAE (dimethylaminoethanol), Bacopa monneiri extract, Sage extract, L-alpha glyceryl phosphoryl choline, Ginko biloba and Ginko biloba extract, Vinpocetine, DHA, nootropics including Phenyltropin, Pikatropin (from Creative Compounds, LLC, Scott City, Mo.), besipirdine, linopirdine, sibopirdine, estrogen and estrogenic compounds, idebenone, T-588 (Toyama Chemical, Japan), and FK960 (Fujisawa Pharmaceutical Co. Ltd.). Compounds described in U.S. Pat. Nos. 5,219,857, 4,904,658, 4,624,954 and 4,665,183 are also useful as cognitive enhancers as described herein. Cognitive enhancers that act through one or more of the above mechanisms are also within the scope of this invention.

In some embodiments, the pharmaceutical formulations described herein are included in kits or packages of pharmaceutical formulations designed for use in the regimens and methods described herein. In some embodiments, these kits are designed for daily oral administration over the specified term or cycle of administration, in some embodiments for the number of prescribed oral administrations per day, and organized so as to indicate a single oral formulation or combination of oral formulations to be taken on each day of the regimen or cycle. In some embodiments, each kit will include oral tablets to be taken on each of the days specified, in some embodiments one oral tablet will contain each of the combined daily dosages indicated and in other embodiments, the administrations of the separate compounds will be present in separate formulations or formulations. In some embodiments, shall have a calendar or days-of-the-week designation directing the administration of the appropriate formulations on the appropriate day or time.

In one embodiment, the pharmaceutical formulations described herein are useful as 5-HT_{1A} receptor antagonists. In another embodiment, the pharmaceutical formulations are useful as 5-HT_{1A} receptor agonists. Compounds that are 5-HT_{1A} antagonists and/or agonists can readily be identified by those skilled in the art using numerous art-recognized methods, including standard pharmacological test procedures such as those described herein. Accordingly, the pharmaceutical formulations described herein are useful for treating a mammal with a 5-HT_{1A}-related disorder. One non-limiting example of a disorder that 5-HT_{1A} receptor antagonists are useful for treating is cognition-related disorder, while a non-limiting example of a disorder that 5-HT_{1A} receptor agonists are useful for treating is anxiety-related disorder. In some embodiments, the pharmaceutical formulations described herein are useful for improving cognitive function or cognitive deficits. Examples of improvements in cognitive function include, without limitation, memory improvement and retention of learned information. Accordingly, the pharmaceutical formulations described herein are useful for slowing the loss of memory and cognition and for maintaining independent function for patients afflicted with a cognition-related disorder. Thus, in one embodiment, the pharmaceutical formulations described herein that contain compounds that act as 5-HT_{1A} receptor antagonists are useful for treating a mammal with a cognition-related disorder. In one embodiment, the pharmaceutical formulations that contain compounds that act as 5-HT_{1A} receptor antagonists are useful for improving the cognitive function of a mammal. Similarly, in one embodiment, pharmaceutical formulations that include compounds that act as 5-HT_{1A} receptor agonists are useful for treating a mammal with an anxiety-related disorder.

In one embodiment, the invention provides a method for treating a 5-HT_{1A}-related disorder, including administering to a mammal in need thereof a pharmaceutical formulation described herein in an amount effective to treat a 5-HT_{1A}-related disorder. In one embodiment, the invention provides a method for treating a cognition-related disorder, including administering to a mammal in need thereof a pharmaceutical formulation described herein in an amount effective to treat a 5-HT_{1A}-related disorder. In one embodiment, the invention provides a method for treating an anxiety-related disorder, including administering to a mammal in need thereof a pharmaceutical formulation described herein in an amount effective to treat a 5-HT_{1A}-related disorder.

In one embodiment, the invention provides a method for treating Alzheimer's disease, including administering to a mammal in need thereof a pharmaceutical formulation described herein in an amount effective to treat Alzheimer's disease. In one embodiment, the invention provides a method for treating symptoms related to or characteristic of Alzheimer's disease, including administering to a mammal in need thereof a pharmaceutical formulation described herein in an amount effective to treat symptoms related to or characteristic of Alzheimer's disease. In one embodiment, the method for treating Alzheimer's disease, or symptoms related to or characteristic of Alzheimer's disease, includes administering a second therapeutic agent. In some embodiments, the second therapeutic agent is an anti-depressant agent, an anti-anxiety agent, an anti-psychotic agent, or a cognitive enhancer.

In one embodiment, the invention provides a method for treating mild cognitive impairment (MCI), includes administering to a mammal in need thereof a compound or a pharmaceutical formulation described herein in an amount effective to treat mild cognitive impairment (MCI). In one embodiment, the method for treating MCI includes administering a second therapeutic agent. In some embodiments, the second therapeutic agent is an anti-depressant agent, an anti-anxiety agent, an anti-psychotic agent, or a cognitive enhancer.

In one embodiment, the invention provides a method for treating depression, including administering to a mammal in need thereof a pharmaceutical formulation described herein in an amount effective to treat depression. In one embodiment, the method for treating depression includes administering a second therapeutic agent. In some embodiments, the second therapeutic agent is an anti-depressant agent, an anti-anxiety agent, an anti-psychotic agent, or a cognitive enhancer.

In one embodiment, the sustained-release tablet formulation described herein are useful for treating sexual dysfunction, e.g. sexual dysfunction associated with drug treatment such as drug treatment with an antidepressant, an antipsychotic, or an anticonvulsant. Accordingly, in one embodiment, the invention provides a method for treating sexual dysfunction associated with drug treatment in a patient in need thereof. The method includes administering to the patient an effective amount of one or more of the formulations disclosed herein. In some embodiments, the drug treatment is antidepressant drug treatment, antipsychotic drug treatment, or anticonvulsant drug treatment.

In certain embodiments, the drug associated with sexual dysfunction is a selective serotonin reuptake inhibitor (SSRI) (for example, fluoxetine, citalopram, escitalopram oxalate, fluvoxamine maleate, paroxetine, or sertraline), a tricyclic antidepressant (for example, desipramine, amitriptyline, amoxipine, clomipramine, doxepin, imipramine, nortriptyline, protriptyline, trimipramine, dothiepin, butriptyline, iprindole, or lofepramine), an aminoketone class compound (for example, bupropion). In some embodiments, the drug is a monoamine oxidase inhibitor (MAOI) (for example, phenelzine, isocarboxazid, or tranylcypromine), a serotonin and norepinepherine reuptake inhibitor (SNRI) (for example, venlafaxine, nefazodone, milnacipran, duloxetine), a norepinephrine reuptake inhibitor (NRI) (for example, reboxetine), a partial 5-HT_{1A} agonist (for example, buspirone), a 5-HT_{2A} receptor antagonist (for example, nefazodone), a typical antipsychotic drug, or an atypical antipsychotic drug. Examples of such antipsychotic drugs include aliphatic phethiazine, a piperazine phenothiazine, a butyrophenone, a substituted benzamide, and a thioxanthine. Additional examples of such drugs include haloperidol, olanzapine, clozapine, risperidone, pimozide, aripiprazol, and ziprasidone. In some cases, the drug is an anticonvulsant, e.g. phenobarbital, phenyloin, primidone, or carbamazepine. In some cases, the patient in need of treatment for sexual dysfunction is being treated with at least two drugs that are antidepressant drugs, antipsychotic drugs, anticonvulsant drugs, or a combination thereof.

In some embodiments of the invention, the sexual dysfunction comprises a deficiency in penile erection.

The invention also provides a method of improving sexual function in a patient in need thereof. The method includes administering to the patient a pharmaceutically effective amount of a pharmaceutical formulation described herein.

The pharmaceutical formulations described herein are also useful in the manufacture of medicaments for treating a 5-HT_{1A}-related disorder in a mammal. Similarly, the pharmaceutical formulations described herein are also useful in the manufacture of medicaments for treating a cognition-related disorder in a mammal. Also, the pharmaceutical formulations described herein are also useful in the manufacture of medicaments for treating an anxiety-related disorder in a mammal.

In some embodiments, the sustained-release tablet formulations described herein include at least one coating on the outermost layer of the tablet. A pharmaceutically acceptable coating agent includes, without limitation, Opadry® White and Opadry® Clear. The coatings can be applied to the formulation using conventional coating methods and equipment. For example, the coatings may be applied to a tablet using a coating pan, an airless spray technique, fluidized bed coating equipment, rotary granulator or the like. In some embodiments, the coating is compressed onto the tablet core. Detailed information concerning materials, equipment and processes for preparing coated dosage forms may be found in Pharmaceutical Dosage Forms: Tablets, Eds. Lieberman et al. (New York: Marcel Dekker, Inc., 1989), and in Ansel et al., Pharmaceutical Dosage Forms and Drug Delivery Systems, 6^{th} Edition (Media, Pa.: Williams & Wilkins, 1995).

In certain embodiments, the present invention provides a method for treating a 5-HT_{1A}-related disorder to a patient in need thereof, the method comprising administering to the patient a therapeutically effective amount of a sustained-release tablet formulation as described hereinabove. In some embodiments, the 5-HT_{1A}-related disorder is a cognition-related disorder or an anxiety-related disorder. In certain other embodiments, the cognition-related disorder is dementia, Parkinson's disease, Huntington's disease, Alzheimer's disease, cognitive deficits associated with Alzheimer's disease, mild cognitive impairment, or schizophrenia. In certain embodiments, the anxiety-related disorder is attention deficit disorder, obsessive compulsive disorder, substance addiction, withdrawal from substance addiction, premenstrual dysphoric disorder, social anxiety disorder, anorexia nervosa, or bulimia nervosa.

In certain embodiments, the present invention provides a process for the preparation of a sustained-release tablet formulation as described hereinabove, which process comprises mixing: a compound of formula (I) or a pharmaceutically acceptably salt thereof, at least one organic acid; at least one release controller; at least one filler; at least one lubricant; followed by pressing into tablet. In certain embodiments, the process may further comprise film coating of the tablet.

Abbreviations

| Abbreviation | Full Description |
|---|---|
| API = | Active Pharmaceutical Ingredient |
| Avg = | Average |
| BRL = | Below Reporting Limits |
| BCS = | Biopharmaceutics Classification System |
| FIM = | First In Man |
| g = | gram |
| HPMC = | Hypromellose |
| hrs = | hours |
| kg = | kilogram |
| mL = | milliliter |
| mm = | millimeter |
| mins = | minutes |
| MCC = | Microcrystalline Cellulose |
| ND = | None Detected |
| NF = | National Formulary |
| PK Blender | Patterson-Kelley Blender |
| RH = | Relative Humidity |
| SD = | Standard Deviation |
| SLS = | Sodium Lauryl Sulfate |
| USP = | United States Pharmacopoeia |

### Methods of Preparation

The general procedure for the preparation of sustained-release tablets involves screening, dry blending, compression, and film coating. Initially, an API (i.e., the compound of formula (I), e.g. trisuccinate salt of 5-fluoro-8-{4-[4-(6-methoxyquinolin-8-yl)piperazin-1-yl]piperidin-1-yl}quinoline), a filler (e.g., microcrystalline cellulose or silicified microcrystalline cellulose), an organic acid (e.g., citric acid anhydrous), and a release controller (e.g. HPMC) are dry blended after screening. Then a lubricant (e.g., magnesium stearate) is screened and added to this blend. This final blend is compressed into tablets. Tablets are then film coated using a coating agent, for example, Opadry® White and Opadry® Clear.

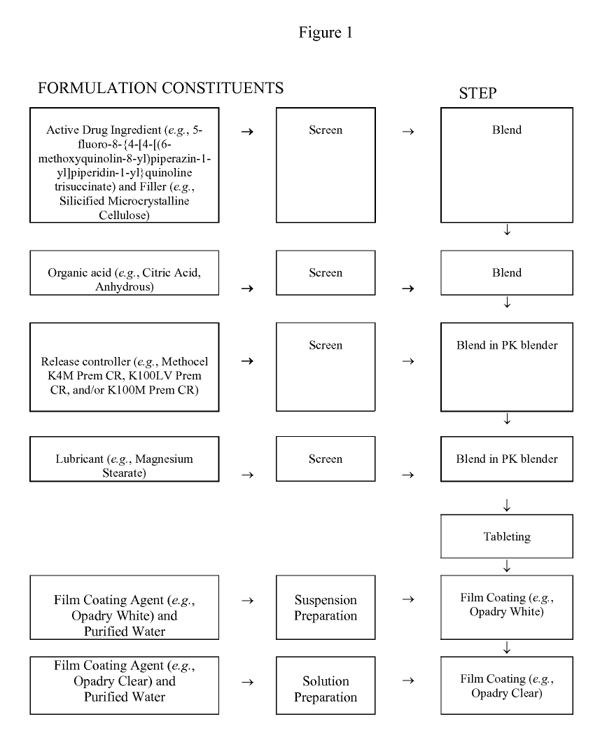

The method for preparing sustained-release tablets is further illustrated in a flow chart as shown in FIG. 1. To achieve better content uniformity, a filler such as microcrystalline cellulose or silicified microcrystalline cellulose is divided into several parts in the manufacturing process based on geometric mixing design.

The following, specific, non-limiting examples are provided to further illustrate the embodiments described herein. The reagents and intermediates used herein are either commercially available or prepared according to standard literature procedures. The materials, methods, and examples presented herein are illustrative and are not intended to limit the scope of the invention.

### EXAMPLES

### Example 1

A sustained-release tablet containing 10 mg of trisuccinate salt of 5-fluoro-8-{4-[4-[(6-methoxyquinolin-8-yl)piperazin-1-yl]piperidin-1-yl}quinoline was prepared with the following constituents:

| | Tablet | Tablet |
|---|---|---|
| Ingredients | Wt/Wt (%) | Weight (mg/tab) |
| Tablet Core | | |
| 5-fluoro-8-{4-[4-(6-methoxyquinolin-8- | 3.86 | 10.00 |
| yl)piperazin-1-yl]piperidin-1- | | |
| yl}quinoline tri-succinate | | |
| SMCC (ProSolv HD90) | 51.80 | 134.00 |
| Citric Acid, Anhydrous | 9.66 | 25.00 |
| HPMC (Methocel K4M Premium CR) | 11.59 | 30.00 |
| HPMC (Methocel K100LV Premium | 19.32 | 50.00 |
| CR) | | |
| Mg Stearate | 0.39 | 1.00 |
| Film Coating | | |
| Opadry White (YS-1-18202A) | 2.90 | 7.50 |
| Opadry Clear (YS-1-19025A) | 0.48 | 1.25 |
| Water, USP, Purified | | |
| TOTAL | 100 | 258.75 |

The process for preparing the above sustained tablet is as follows:

(A) Blending and Compression:
1. Screen required amounts of ProSolv HD 90 and 5-fluoro-8-{4-[4-(6-methoxyquinolin-8-yl)piperazin-1-yl]piperidin-1-yl}quinoline tri-succinate through a 20-mesh screen and mix them in a plastic bag.
2. Screen Citric Acid Anhydrous through a 20-mesh screen, and mix with the blend from step#1. Transfer the resulting blend to an appropriate PK blender.
3. Screen remaining ProSolv HD90 through a 20-mesh screen into the PK blender from step#2 and mix for 5 minutes without an intensifier bar.
4. Screen HPMC through a 20-mesh screen into the PK blender from step #3 and mix for 20 minutes without an intensifier bar.
5. Screen Magnesium Stearate through a 30-mesh screen and blend with an approximately equal portion of the blend from step #4 and add to the blend in step #4. Mix for 2 minutes without an intensifier bar.
6. Compress the resulting blend in tablets as per the specifications.

(B) Film Coating:
1. Apply sufficient Opadry® White suspension to achieve an average weight gain of 7.5 mg/tablet utilizing suggested coating parameters.
2. Apply sufficient Opadry® Clear solution to achieve an average weight gain of 1.25 mg/tablet utilizing suggested coating parameters.
3. Cool the tablets to approximately 25° C. Unload the tablets from coating pan and store in the light-resistant, poly-lined containers or equivalent.

### Dissolution Testing

Dissolution testing was conducted using USP Apparatus 2 at 75 rpm under two different conditions. Apparatus 2 described in the United States Pharmacopoeia (USP29-NF24, page 2673) having a rotation speed of 75 rpm, and a dissolution medium at 37° C. Under the first condition, a single stage dissolution method is employed using pH 4.5 buffer and a paddle speed of 75 rpm. Under the second condition, a two-stage dissolution method is utilized, in which tablets are subjected to pH 1 buffer for two hours followed by submersion in pH 6.5 buffer with 1% SLS for an additional 6 hours.

Results obtained in the dissolution tests are shown in Tables 1 and 2.

TABLE 1
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 13.08 |
| 1 | 21.16 |
| 2 | 33.99 |
| 4 | 53.51 |
| 6 | 67.74 |
| 8 | 78.74 |

TABLE 2
Dissolution profile of 5-fluoro-8-{4-[4-[(6-methoxyquinolin-
8-yl)piperazin-1-yl]piperidin-1-yl}quinoline trisuccinate
tablet in two-stage dissolution

| Time (hr.) | % Of API Released |
|---|---|
| 0 | 0.00 |
| 0.5 | 22.88 |
| 1 | 34.50 |
| 2 | 50.90 |
| 4 | 64.65 |
| 6 | 74.99 |
| 8 | 82.42 |

### Example 2

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the constituents shown below:

| | Tablet | Tablet |
|---|---|---|
| Ingredients | Wt/Wt (%) | Weight (mg/tab) |
| Tablet Core | | |
| 5-fluoro-8-{4-[4-(6- | 3.86 | 10.00 |
| methoxyquinolin-8-yl)piperazin-1- | | |
| yl]piperidin-1-yl}quinoline tri- | | |
| succinate | | |
| SMCC (ProSolv HD90) | 34.40 | 89.00 |
| Citric Acid, Anhydrous | 9.66 | 25.00 |
| HPMC (Methocel K4M Premium | 19.32 | 50.00 |
| CR) | | |
| HPMC (Methocel K100M | 28.99 | 75.00 |
| Premium CR) | | |
| Mg Stearate | 0.39 | 1.00 |
| Film Coating | | |
| Opadry ® White (YS-1-18202A) | 2.90 | 7.50 |
| Opadry ® Clear (YS-1-19025A) | 0.48 | 1.25 |
| TOTAL | 100 | 258.75 |

Dissolution testing was carried out as described in Example 1 and gave results shown in Tables 3 and 4.

TABLE 3
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 2.44 |
| 1 | 11.29 |
| 2 | 18 |
| 4 | 30.91 |
| 6 | 40.78 |
| 8 | 48.54 |

TABLE 4
Dissolution profile of 5-fluoro-8-{4-[4-[(6-methoxyquinolin-
8-yl)piperazin-1-yl]piperidin-1-yl}quinoline trisuccinate
tablet in two-stage dissolution

| Time (hr.) | % Of API Released |
|---|---|
| 0 | 0.00 |
| 0.5 | 16.71 |
| 1 | 25.22 |
| 2 | 37.88 |
| 4 | 48.07 |
| 6 | 56.49 |
| 8 | 62.71 |

### Example 3

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the following constituents:

| Ingredients | Tablet |
|---|---|
| Tablet Core | Weight (mg/tab) |
| 5-fluoro-8-{4-[4-(6-methoxyquinolin-8- | 10.0 |
| yl)piperazin-1-yl]piperidin-1-yl}quinoline tri- | |
| succinate | |
| SMCC (ProSolv HD90) | 154.0 |
| Citric Acid, Anhydrous | 25.0 |
| HPMC Methocel K100 LV CR) | 60.0 |
| Mg Stearate | 1.00 |
| TOTAL | 250 |

Dissolution testing was carried out as described in Example 1 and gave results shown in Table 5.

TABLE 5
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 16.93 |
| 1 | 27.67 |
| 2 | 48.73 |
| 4 | 85.53 |
| 6 | 96.83 |
| 8 | 98.70 |
| 9 | 97.90 |

### Example 4

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the constituents as shown below:

| Ingredients | Tablet |
|---|---|
| Tablet Core | Weight (mg/tab) |
| 5-fluoro-8-{4-[4-(6-methoxyquinolin-8- | 10.0 |
| yl)piperazin-1-yl]piperidin-1-yl}quinoline | |
| tri-succinate | |
| SMCC (ProSolv HD90) | 139.0 |
| Citric Acid, Anhydrous | 25.0 |
| HPMC Methocel K100 LV CR) | 75.0 |
| Mg Stearate | 1.00 |
| TOTAL | 250 |

Dissolution testing was carried out as described in Example 1 and gave results shown in Table 6.

TABLE 6
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 19.53 |
| 1 | 30.80 |
| 2 | 49.70 |
| 4 | 85.70 |
| 6 | 92.97 |
| 8 | 95.30 |
| 9 | 96.40 |

### Example 5

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the following constituents:

| Ingredients | Tablet |
|---|---|
| Tablet Core | Weight (mg/tab) |
| 5-fluoro-8-{4-[4-(6-methoxyquinolin-8- | 10.0 |
| yl)piperazin-1-yl]piperidin-1-yl}quinoline tri- | |
| succinate | |
| SMCC (ProSolv HD90) | 104.0 |
| Citric Acid, Anhydrous | 25.0 |
| HPMC Methocel K100 LV CR) | 60.0 |
| Fast-Flow Lactose | 50.0 |
| Mg Stearate | 1.00 |
| TOTAL | 250 |

Dissolution testing was carried out as described in Example 1 and gave results shown in Table 7.

TABLE 7
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 23.57 |
| 1 | 36.50 |
| 2 | 60.10 |
| 4 | 80.57 |
| 6 | 94.33 |
| 8 | 93.83 |
| 9 | 93.57 |

### Example 6

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the constituents as shown below:

| Ingredients | Tablet |
|---|---|
| Tablet Core | Weight (mg/tab) |
| 5-fluoro-8-{4-[4-(6-methoxyquinolin-8-yl)piperazin- | 10.0 |
| 1-yl]piperidin-1-yl}quinoline tri-succinate | |
| SMCC (ProSolv HD90) | 101.5 |
| Citric Acid, Anhydrous | 25.0 |
| HPMC Methocel K4M CR | 62.5 |
| HPMC Methocel K100 LV CR) | 50.0 |
| Mg Stearate | 1.00 |
| TOTAL | 250 |

Dissolution testing was carried out as described in Example 1 and gave results shown in Tables 8 and 9.

TABLE 8
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 8.2 |
| 1 | 14.97 |
| 2 | 25.43 |
| 4 | 42.77 |
| 6 | 53.73 |
| 8 | 64.57 |

TABLE 9
Dissolution profile of 5-fluoro-8-{4-[4-[(6-methoxyquinolin-
8-yl)piperazin-1-yl]piperidin-1-yl}quinoline trisuccinate
tablet in two-stage dissolution

| Time (hr.) | % Of API Released |
|---|---|
| 0 | 0.00 |
| 0.5 | 19.16 |
| 1 | 28.62 |
| 2 | 42.28 |
| 4 | 54.48 |
| 6 | 64.72 |
| 8 | 71.52 |

### Example 7

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the following constituents:

| Ingredients | Tablet |
|---|---|
| Tablet Core | Weight (mg/tab) |
| 5-fluoro-8-{4-[4-(6-methoxyquinolin-8-yl)piperazin- | 10.0 |
| 1-yl]piperidin-1-yl}quinoline tri-succinate | |
| SMCC (ProSolv HD90) | 114.0 |
| Citric Acid, Anhydrous | 25.0 |
| HPMC Methocel K4M CR | 100.0 |
| Mg Stearate | 1.00 |
| TOTAL | 250 |

Dissolution testing was carried out as described in Example 1 and gave results shown in Tables 10 and 11.

TABLE 10
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 6.57 |
| 1 | 13.55 |
| 2 | 21.56 |
| 4 | 37.24 |
| 6 | 47.13 |
| 8 | 56.96 |

TABLE 11
Dissolution profile of 5-fluoro-8-{4-[4-[(6-methoxyquinolin-
8-yl)piperazin-1-yl]piperidin-1-yl}quinoline trisuccinate
tablet in two-stage dissolution

| Time (hr.) | % Of API Released |
|---|---|
| 0 | 0.00 |
| 0.5 | 19.49 |
| 1 | 28.90 |
| 2 | 42.62 |
| 4 | 54.25 |
| 6 | 64.04 |
| 8 | 71.08 |

### Example 8

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the constituents as shown below:

| | Tablet |
|---|---|
| Ingredients | Weight (mg/tab) |
| Tablet Core | |
| 5-fluoro-8-{4-[4-(6-methoxyquinolin-8-yl)piperazin- | 10.0 |
| 1-yl]piperidin-1-yl}quinoline tri-succinate | |
| SMCC (ProSolv HD90) | 89.0 |
| Citric Acid, Anhydrous | 25.0 |
| HPMC Methocel K4M CR | 125.0 |
| Mg Stearate | 1.00 |
| TOTAL | 250 |

Dissolution testing was carried out as described in Example 1 and gave results shown in Tables 12 and 13.

TABLE 12
Dissolution profile of 5-fluoro-8-
{4-[4-[(6-methoxyquinolin-8-yl)piperazin-1-yl]
piperidin-1-yl}qiunoline trisuccinate tablet
in one-stage dissolution

| Time (hr.) | % Of API Released |
|---|---|
| 0 | 0.00 |
| 0.5 | 5.41 |
| 1 | 12.66 |
| 2 | 20.82 |
| 4 | 34.73 |
| 6 | 45.61 |
| 8 | 54.60 |

TABLE 13
Dissolution profile of 5-fluoro-8-{4-[4-[(6-methoxyquinolin-
8-yl)piperazin-1-yl]piperidin-1-yl}quinoline trisuccinate
tablet in two-stage dissolution

| Time (hr.) | % Of API Released |
|---|---|
| 0 | 0.00 |
| 0.5 | 17.98 |
| 1 | 27.18 |
| 2 | 40.61 |
| 4 | 52.92 |
| 6 | 63.02 |
| 8 | 70.18 |

### Example 9

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the following constituents:

| Ingredients | Tablet |
|---|---|
| Tablet Core | Weight (mg/tab) |
| 5-fluoro-8-{4-[4-(6-methoxyquinolin-8-yl)piperazin- | 10.0 |
| 1-yl]piperidin-1-yl}quinoline tri-succinate | |
| SMCC (ProSolv HD90) | 89.0 |
| Citric Acid, Anhydrous | 25.0 |
| HPMC Methocel K100M CR | 125.0 |
| Mg Stearate | 1.00 |
| TOTAL | 250 |

Dissolution testing was carried out as described in Example 1 and gave results shown in Tables 14 and 15.

TABLE 14
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 6.09 |
| 1 | 11.27 |
| 2 | 17.1 |
| 4 | 29.44 |
| 6 | 32.39 |
| 8 | 46.61 |

TABLE 15
Dissolution profile of 5-fluoro-8-{4-[4-[(6-methoxyquinolin-
8-yl)piperazin-1-yl]piperidin-1-yl}quinoline trisuccinate
tablet in two-stage dissolution

| Time (hr.) | % Of API Released |
|---|---|
| 0 | 0.00 |
| 0.5 | 17.31 |
| 1 | 25.89 |
| 2 | 38.05 |
| 4 | 47.99 |
| 6 | 55.56 |
| 8 | 61.29 |

### Example 10

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the constituents as shown below:

| Ingredients | Tablet |
|---|---|
| Tablet Core | Weight (mg/tab) |
| 5-fluoro-8-{4-[4-(6-methoxyquinolin-8-yl)piperazin- | 10.0 |
| 1-yl]piperidin-1-yl}quinoline tri-succinate | |
| SMCC (ProSolv HD90) | 109.0 |
| Citric Acid, Anhydrous | 5.0 |
| HPMC Methocel K100M CR | 125.0 |
| Mg Stearate | 1.00 |
| TOTAL | 250 |

Dissolution testing was carried out as described in Example 1 and gave results shown in Table 16.

TABLE 16
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 3.77 |
| 1 | 6.51 |
| 2 | 11.01 |
| 4 | 16.84 |
| 6 | 22.29 |
| 8 | 26.49 |

### Example 11

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the following constituents:

| Ingredients | Tablet |
|---|---|
| Tablet Core | Weight (mg/tab) |
| 5-fluoro-8-{4-[4-(6-methoxyquinolin-8-yl)piperazin- | 10.0 |
| 1-yl]piperidin-1-yl}quinoline tri-succinate | |
| SMCC (ProSolv HD90) | 109.0 |
| Citric Acid, Anhydrous | 25.0 |
| HPMC Methocel K4M CR | 55.0 |
| HPMC Methocel K100 LV CR) | 50.0 |
| Mg Stearate | 1.00 |
| TOTAL | 250 |

Dissolution testing was carried out as described in Example 1 and gave results shown in Table 17.

TABLE 17
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 8.73 |
| 1 | 18.94 |
| 2 | 28.91 |
| 4 | 43.15 |
| 6 | 58.57 |
| 8 | 71.41 |

### Example 12

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the constituents as shown below:

| Ingredients | Tablet |
|---|---|
| Tablet Core | Weight (mg/tab) |
| 5-fluoro-8-{4-[4-(6-methoxyquinolin-8- | 10.0 |
| yl)piperazin-1-yl]piperidin-1-yl}quinoline | |
| tri-succinate | |
| SMCC (ProSolv HD90) | 109.0 |
| Citric Acid, Anhydrous | 25.0 |
| HPMC Methocel K4M CR | 45.0 |
| HPMC Methocel K100 LV CR) | 60.0 |
| Mg Stearate | 1.00 |
| TOTAL | 250 |

Dissolution testing was carried out as described in Example 1 and gave results shown in Table 18.

TABLE 18
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 5.95 |
| 1 | 16.32 |
| 2 | 25.62 |
| 4 | 40.52 |
| 6 | 54.22 |
| 8 | 66.81 |

### Example 13

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the following constituents:

| Ingredients | Tablet |
|---|---|
| Tablet Core | Weight (mg/tab) |
| 5-fluoro-8-{4-[4-(6-methoxyquinolin-8- | 10.0 |
| yl)piperazin-1-yl]piperidin-1-yl}quinoline tri- | |
| succinate | |
| SMCC (ProSolv HD90) | 116.5 |
| Citric Acid, Anhydrous | 25.0 |
| HPMC Methocel K4M CR | 37.5 |
| HPMC Methocel K100 LV CR) | 60.0 |
| Mg Stearate | 1.00 |
| TOTAL | 250 |

Dissolution testing was carried out as described in Example 1 and gave results shown in Table 19.

TABLE 19
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 7.61 |
| 1 | 17.72 |
| 2 | 30.36 |
| 4 | 47.31 |
| 6 | 63.75 |
| 8 | 76.14 |

### Example 14

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the constituents as shown below:

| Ingredients | Tablet |
|---|---|
| Tablet Core | Weight (mg/tab) |
| 5-fluoro-8-{4-[4-(6-methoxyquinolin-8-yl)piperazin- | 10.0 |
| 1-yl]piperidin-1-yl}quinoline tri-succinate | |
| SMCC (ProSolv HD90) | 126.5 |
| Citric Acid, Anhydrous | 25.0 |
| HPMC Methocel K4M CR | 37.5 |
| HPMC Methocel K100 LV CR) | 50.0 |
| Mg Stearate | 1.00 |
| TOTAL | 250 |

Dissolution testing was carried out as described in Example 1 and gave results shown in Tables 20 and 21.

TABLE 20
Dissolution profile of 5-fluoro-8-{4-[4-[(6-methoxyquinolin-
8-yl)piperazin-1-yl]piperidin-1-yl}quinoline trisuccinate
tablet in one-stage dissolution

| Time (hr.) | % Of API Released |
|---|---|
| 0 | 0.00 |
| 0.5 | 8.11 |
| 1 | 19.62 |
| 2 | 32.19 |
| 4 | 48.83 |
| 6 | 65.94 |
| 8 | 77.4 |

TABLE 21
Dissolution profile of 5-fluoro-8-{4-[4-[(6-methoxyquinolin-
8-yl)piperazin-1-yl]piperidin-1-yl}quinoline trisuccinate
tablet in two-stage dissolution

| Time (hr.) | % Of API Released |
|---|---|
| 0 | 0.00 |
| 0.5 | 22.12 |
| 1 | 33.63 |
| 2 | 49.41 |
| 4 | 62.34 |
| 6 | 72.01 |
| 8 | 80.05 |

### Example 15

A sustained-release tablet containing 10 mg of trisuccinate salt of 5-fluoro-8-{4-[4-(6-methoxyquinolin-8-yl)piperazin-1-yl]piperidin-1-yl}quinoline was prepared according to the process described in Example 1 with the following constituents:

| Ingredients | Tablet |
|---|---|
| Tablet Core | Weight (mg/tab) |
| 5-fluoro-8-{4-[4-(6-methoxyquinolin-8-yl)piperazin- | 10.0 |
| 1-yl]piperidin-1-yl}quinoline tri-succinate | |
| SMCC (ProSolv HD90) | 141.5 |
| Citric Acid, Anhydrous | 25.0 |
| HPMC Methocel K4M CR | 22.5 |
| HPMC Methocel K100 LV CR) | 50.0 |
| Mg Stearate | 1.00 |
| TOTAL | 250 |

Dissolution testing was carried out as described in Example 1 and gave results shown in Table 22.

TABLE 22
Dissolution profile of 5-fluoro-8-{4-[4-[(6-methoxyquinolin-
8-yl)piperazin-1-yl]piperidin-1-yl}quinoline trisuccinate
tablet in two-stage dissolution

| Time (hr.) | % Of API Released |
|---|---|
| 0 | 0.00 |
| 0.5 | 26.23 |
| 1 | 38.53 |
| 2 | 55.61 |
| 4 | 69.31 |
| 6 | 79.41 |
| 8 | 87.24 |

### Example 16

### In Vivo Testing

Eight male dogs (10.2-13.4 kg) were divided into two groups, four dogs per group. Group 1 received the tablet formulation of Example 1 and Group 2 received the tablet formulation of Example 2. All formulations were administered following an overnight fast. The following pharmacokinetic parameters were determined for each dog, and descriptive statistics were calculated: AUC, Cₘₐₓ, tₘₐₓ and t_{1/2}, which have been summarized in Table 23.

TABLE 23
Pharmacokinetic parameters following a single oral
administration of sustained-release tablet formulations
of Examples 1 and 2 to Male Beagle dogs

| Mean (SD) | | |
|---|---|---|
| | Example 1 | Example 2 |

| | | | | |
|---|---|---|---|---|
| Dose | 0.88 | (0.05) | 0.88 | (0.07) |
| (mg/kg) | | | | |
| Cₘₐₓ | 30.4 | (19.1) | 40.5 | (33.5) |
| (ng/mL) | | | | |
| AUC₀₋ₜ^{a} | 212 | (138) | 294 | (256) |
| (ng · hr/mL) | | | | |
| AUC_{0-∞} | 318 | (128) | 106 | (NC) |
| (ng · hr/mL) | | | | |
| Cₘₐₓ/Dose | 35.5 | (22.9) | 46.6 | (37.3) |
| AUC₀₋ₜ/Dose | 248 | (165) | 336 | (283) |
| (mg/kg) | | | | |
| tₘₐₓ | 2.3 | (1.3) | 1.9 | (1.5) |
| (hr) | | | | |
| t_{1/2} | 13.1 | (5.1) | 11.4 | (NC) |
| (hr) | | | | |

^{a}Value represents the area until the last observed concentration-time point
NC = Not Calculated

We claim:

1. A sustained-release tablet formulation for oral administration comprising:
(a) a compound of formula (I), or a pharmaceutically acceptable salt thereof, 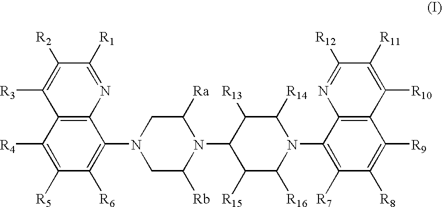
wherein R₁, R₂, R₃, R₄, R₅, R₆, R₇, R₈, R₉, R₁₀, R₁₁, R₁₂, R₁₃, R₁₄, R₁₅, and R₁₆, are each independently —H, (C₁-C₆)-alkyl, (C₁-C₆)-haloalkyl, (C₂-C₆)-alkenyl, or (C₂-C₆)-alkynyl, halogen, —CF₃, —NO₂, —CN, —OR₂₅, —OSO₂R₂₅, —SR₂₅, —SO₂R₂₅, —SO₂N(R₂₅)₂, —N(R₂₅)₂, C(O), —COR₂₅, —CO₂R₂₅, —NR₂₅CO₂R₂₅, —NR₂₅COR₂₅, —NR₂₅CON(R₂₅)₂, or —CON(R₂₅)₂;
Rₐ and R_{b} are each independently —H or —CH₃; and
R₂₅ is —H, linear or branched (C₁-C₆)-alkyl, (C₁-C₆)-haloalkyl, (C₂-C₆)-alkenyl, or (C₂-C₆)-alkynyl;
(b) at least one organic acid;
(c) at least one release controller;
(d) at least one filler; and
(e) at least one lubricant.

2. The formulation of claim 1, wherein R₅ is —H, (C₁-C₆)-alkyl, —OR₂₅, halogen, or —CF₃; and R₉ is —H, (C₁-C₆)-alkyl, —OR₂₅, halogen, —CF₃, —NO₂, or —CN.

3. The formulation of claim 1, wherein R₁₋₄ and R₆₋₁₆ are each H; R₅ is —OR₂₅; R₂₅ is (C₁-C₆)-alkyl; and R₉ is halogen.

4. The formulation of claim 1, wherein the compound of formula (I) is selected from the group consisting of:
6-methoxy-8-[4-(1-quinolin-8-yl-piperidin-4-yl)-piperazin-1-yl]-quinoline;
6-fluoro-8-[4-(1-quinolin-8-yl-piperidin-4-yl)-piperazin-1-yl]-quinoline;
5-fluoro-8-[4-(1-quinolin-8-yl-piperidin-4-yl)-piperazin-1-yl]-quinoline;
7-fluoro-8-(4-(4-(6-methoxyquinolin-8-yl)piperazin-1-yl)piperidin-1-yl)quinoline;
6-fluoro-8-{4-[1-(8-fluoroquinolin-7-yl)piperidin-4-yl]piperazin-1-yl}quinoline;
3-trifluoromethyl-8-(4-(4-(6-methoxyquinolin-8-yl)piperazin-1-yl)piperidin-1-yl)quinoline;
6-methoxy-8-(4-(1-(quinolin-8-ylmethyl)piperidin-4-yl)piperazin-1-yl)quinoline;
5-fluoro-4-methoxy-8-(4-(4-(6-methoxyquinolin-8-yl)piperazin-1-yl)piperidin-1-yl)-2-(trifluoromethyl)quinoline;
5-fluoro-8-(4-(4-(6-methoxyquinolin-8-yl)piperazin-1-yl)piperidin-1-yl)quinoline;
5-fluoro-8-(4-(4-(6-methoxyquinolin-8-yl)piperazin-1-yl)piperidin-1-yl)quinoline trisuccinate;
8-[4-(1-quinolin-8-yl-piperidin-4-yl)-piperazin-1-yl]-quinoline;
6-chloro-8-[4-(4-(6-chloro)-quinolin-8-yl-piperidin-1-yl)-piperazin-1-yl]-quinoline;
6-fluoro-8-[4-(4-(6-chloro)-quinolin-8-yl-piperidin-1-yl)-piperazin-1-yl]-quinoline;
5-chloro-8-[4-(1-quinolin-8-yl-piperidin-4-yl)-piperazin-1-yl]-quinoline;
2-methyl-8-[4-(1-quinolin-8-yl-piperidin-4-yl)-piperazin-1-yl]-quinoline;
6-chloro-8-[4-(1-quinolin-8-yl-piperidin-4-yl)-piperazin-1-yl]-quinoline;
8-[4-(1-quinolin-8-yl-piperidin-4-yl)-piperazin-1-yl]-5-trifluoromethyl-quinoline;
5-methoxy-8-[4-(1-quinolin-8-yl-piperidin-4-yl)-piperazin-1-yl]-quinoline;
5-fluoro-8-[4-(4-quinolin-8-yl-piperazin-1-yl)-piperidin-1-yl]-quinoline;
6-methoxy-8-[4-(2-methylquinolin-8-yl-piperidin-4-yl)-piperazin-1-yl]-quinoline;
6-fluoro-8-(4-(1-(2-methylquinolin-8-yl)piperidin-4-yl)piperazin-1-yl)quinoline;
6-methoxy-8-[4-(3-methylquinolin-8-yl-piperidin-4-yl)-piperazin-1-yl]-quinoline;
6-methoxy-8-(4-(1-(4-methylquinolin-8-yl)piperidin-4-yl)piperazin-1-yl)quinoline;
6-methoxy-8-(4-(1-(2,4-dimethylquinolin-8-yl)piperidin-4-yl)piperazin-1-yl)quinoline;
6-methoxy-8-(4-(1-(2,4-dimethyl-5-fluoroquinolin-8-yl)piperidin-4-yl)piperazin-1-yl)quinoline;
6-methoxy-8-(4-(1-(2-(trifluoromethyl)quinolin-8-yl)piperidin-4-yl)piperazin-1-yl)quinoline;
6-fluoro-8-(4-(1-(5-fluoroquinolin-8-yl)piperidin-4-yl)piperazin-1-yl)quinoline;
6-methoxy-8-(4-(1-(6-bromoquinolin-8-yl)piperidin-4-yl)piperazin-1-yl)quinoline;
6-methoxy-8-(4-(1-(6-fluoroquinolin-8-yl)piperidin-4-yl)piperazin-1-yl)quinoline;
6-fluoro-8-(4-(1-(7-fluoroquinolin-8-yl)piperidin-4-yl)piperazin-1-yl)quinoline;
6-methoxy-8-{4-[1-(8-fluoroquinolin-7-yl)piperidin-4-yl]piperazin-1-yl}quinoline;
6-methoxy-8-{4-[1-(2-trifluoromethyl-4-methoxyquinolin-7-yl)piperidin-4-yl]piperazin-1-yl}quinoline;
6-methoxy-8-(4-(1-(2-trifluoromethyl-4-methoxyquinolin-8-yl)piperidin-4-yl)piperazin-1-yl)quinoline;
5-fluoro-8-(4-(4-(6-methoxyquinolin-8-yl)piperazin-1-yl)piperidin-1-yl)-2-trifluoromethylquinoline;
5-fluoro-8-(4-(4-(6-methoxyquinolin-8-yl)piperazin-1-yl)piperidin-1-yl)-3-trifluoromethylquinoline;
5-fluoro-8-(4-(4-(6-methoxyquinolin-8-yl)piperazin-1-yl)piperidin-1-yl)-4-trifluoromethylquinoline;
2,5-difluoro-8-(4-(4-(6-methoxyquinolin-8-yl)piperazin-1-yl)piperidin-1-yl)quinoline;
3,5-difluoro-8-(4-(4-(6-methoxyquinolin-8-yl)piperazin-1-yl)piperidin-1-yl)quinoline; and
4,5-difluoro-8-(4-(4-(6-methoxyquinolin-8-yl)piperazin-1-yl)piperidin-1-yl)quinoline.

5. The formulation of claim 1, wherein the compound of formula (I) is 5-fluoro-8-{4-[4-(6-methoxyquinolin-8-yl)piperazin-1-yl]piperidin-1-yl}quinoline trisuccinate.

6. The formulation of claim 1, characterized in that the formulation releases at least about 25% by weight of the compound of formula (I) or a pharmaceutically acceptable salt thereof, after 8 hours in a dissolution test apparatus at a rotation speed of 75 rpm, and a single stage dissolution medium containing pH 4.5 buffer at 37° C.

7. The formulation of claim 1, characterized in that the formulation releases at least about 25% by weight of the compound of formula (I) or a pharmaceutically acceptable salt thereof, after 8 hours in Apparatus 2 described in the United States Pharmacopoeia (USP29-NF24, page 2673) having a rotation speed of 75 rpm, and a single stage dissolution medium containing pH 4.5 buffer at 37° C.

8. The formulation of claim 1, characterized in that the formulation releases at least about 45% by weight of the compound of formula (I) or a pharmaceutically acceptable salt thereof, after 8 hours in a dissolution test apparatus at a rotation speed of 75 rpm, and a single stage dissolution medium containing pH 4.5 buffer at 37° C.

9. The formulation of claim 1, characterized in that the formulation releases at least about 45% by weight of the compound of formula (I) or a pharmaceutically acceptable salt thereof, after 8 hours in Apparatus 2 described in the United States Pharmacopoeia (USP29-NF24, page 2673) having a rotation speed of 75 rpm, and a single stage dissolution medium containing pH 4.5 buffer at 37° C.

10. The formulation of claim 7, wherein at least 15% by weight of the compound of formula (I) or a pharmaceutically acceptable salt thereof, is released after 2 hours.

11. The formulation of claim 7, wherein less than 40% by weight of the compound of formula (I) or a pharmaceutically acceptable salt thereof, is released after 2 hours.

12. The formulation of claim 7, wherein less than 20% by weight of the compound of formula (I) or a pharmaceutically acceptable salt thereof, is released after 2 hours.

13. The formulation of claim 7, wherein less than 60% by weight of the compound of formula (I) or a pharmaceutically acceptable salt thereof, is released after 4 hours.

14. The formulation of claim 7, wherein less than 40% by weight of the compound of formula (I) or a pharmaceutically acceptable salt thereof, is released after 4 hours.

15. The formulation of claim 7, wherein less than 70% by weight of the compound of formula (I) or a pharmaceutically acceptable salt thereof, is released after 6 hours.

16. The formulation of claim 7, wherein less than 50% by weight of the compound of formula (I) or a pharmaceutically acceptable salt thereof, is released after 6 hours.

17. The formulation of claim 1, characterized in that the formulation releases at least 60% by weight of the compound of formula (I) or a pharmaceutically acceptable salt thereof, after 8 hours in a dissolution test apparatus at a rotation speed of 75 rpm, and a two stage dissolution medium containing (i) pH 1 buffer at 37° C. for two hours, and followed by (ii) pH 6.5 buffer with 1% SLS for additional 6 hours at 37° C.

18. The formulation of claim 1, characterized in that the formulation releases at least 60% by weight of the compound of formula (I) or a pharmaceutically acceptable salt thereof, after 8 hours in Apparatus 2 described in the United States Pharmacopoeia (USP29-NF24, page 2673) having a rotation speed of 75 rpm, and a two stage dissolution medium containing (i) pH 1 buffer at 37° C. for two hours, and followed by (ii) pH 6.5 buffer with 1% SLS for additional 6 hours at 37° C.

19. The formulation of claim 17, wherein at least 35% by weight of the compound of formula (I) or a pharmaceutically acceptable salt thereof, is released after 2 hours.

20. The formulation of claim 17, wherein less than 55% by weight of the compound of formula (I) or a pharmaceutically acceptable salt thereof, is released after 2 hours.

21. The formulation of claim 17, wherein less than 40% by weight of the compound of formula (I) or a pharmaceutically acceptable salt thereof, is released after 2 hours.

22. The formulation of claim 17, wherein less than 70% by weight of the compound of formula (I) or a pharmaceutically acceptable salt thereof, is released after 4 hours.

23. The formulation of claim 17, wherein less than 50% by weight of the compound of formula (I) or a pharmaceutically acceptable salt thereof, is released after 4 hours.

24. The formulation of claim 17, wherein less than 80% by weight of the compound of formula (I) or a pharmaceutically acceptable salt thereof, is released after 6 hours.

25. The formulation of claim 17, wherein less than 60% by weight of the compound of formula (I) or a pharmaceutically acceptable salt thereof, is released after 6 hours.

26. The formulation of claim 1, wherein the organic acid is citric acid, ascorbic acid, aspartic acid, glutamic acid, tartaric acid, succinic acid, malic acid, erythorbic acid, propionic acid, lactic acid, oleic acid, fumaric acid, benzoic acid, or alginic acid.

27. The formulation of claim 1, wherein the release controller is hydroxypropyl methylcellulose, hydroxypropyl cellulose, hydroxyethyl cellulose, or hydroxypropyl methylcellulose phthalate.

28. The formulation of claim 1, wherein the filler is microcrystalline cellulose, silicified microcrystalline cellulose, lactose, calcium carbonate, calcium sulfate, calcium phosphate, sodium chloride, maltodextrin, dextrose, fructose, maltose, mannitol, starch, sucrose, or kaolin.

29. The formulation of claim 1, wherein the lubricant is magnesium stearate, calcium stearate, stearic acid, talc, hydrogenated vegetable oils, polyethylene glycol, or colloidal silicon dioxide.

30. The formulation of claim 1, wherein the organic acid is citric acid, the release controller is hydroxypropyl methylcellulose, the filler is silicified microcrystalline cellulose, and the lubricant is magnesium stearate.

31. The formulation of claim 1, wherein the release controller is one or more HPMC selected from Methocel® K4M Premium CR, Methocel® K100M Premium CR, and Methocel® K100LV Premium CR.

32. The formulation of claim 1, wherein the formulation contains from about 0.1 mg to about 100 mg of the compound of formula (I), or a pharmaceutically acceptable salt thereof.

33. The formulation of claim 1, wherein the formulation contains from about 0.5 mg to about 25 mg of the compound of formula (I), or a pharmaceutically acceptable salt thereof.

34. The formulation of claim 1, wherein the compound of formula (I), or a pharmaceutically acceptable salt thereof, makes up about 0.02% to about 20% by weight of the formulation, expressed in the form of its free base.

35. The formulation of claim 1, wherein the organic acid makes up about 2% to about 20% by weight of the formulation.

36. The formulation of claim 1, wherein the organic acid makes up about 5% to about 15% by weight of the formulation.

37. The formulation of claim 1, wherein the release controller makes up about 10% to about 60% by weight of the formulation.

38. The formulation of claim 1, wherein the release controller makes up about 30% to about 50% by weight of the formulation.

39. The formulation of claim 1, wherein the filler makes up about 25% to about 65% by weight of the formulation.

40. The formulation of claim 1, wherein the lubricant makes up about 0.1% to about 5% by weight of the formulation.

41. A method for treating a 5-HT_{1A}-related disorder to a patient in need thereof, the method comprising administering to the patient a therapeutically effective amount of a sustained-release tablet formulation as defined in claim 1.

42. The method of claim 41, wherein the 5-HT_{1A}-related disorder is a cognition-related disorder or an anxiety-related disorder.

43. The method of claim 42, wherein the cognition-related disorder is dementia, Parkinson's disease, Huntington's disease, Alzheimer's disease, cognitive deficits associated with Alzheimer's disease, mild cognitive impairment, or schizophrenia.

44. The method of claim 42, wherein the anxiety-related disorder is attention deficit disorder, obsessive compulsive disorder, substance addiction, withdrawal from substance addiction, premenstrual dysphoric disorder, social anxiety disorder, anorexia nervosa, or bulimia nervosa.

45. A process for the preparation of a sustained-release tablet formulation as defined in claim 1, which process comprises mixing: a compound of formula (I) or a pharmaceutically acceptably salt thereof, at lease one organic acid; at least one release controller; at least one filler; at least one lubricant; followed by pressing into tablet.

46. The process of claim 45, further comprising film coating of the tablet.

47. The use of a formulation as described in claim 46, in the manufacture of a medicament for treating a 5HT_{1A}-related disorder.
